SEQUENCE LISTING (34 sequences)

SEQ ID NO: 1 - RNA, 25 nt, synthetic construct
  source 1..25: mol_type = other RNA
cctcctcgcctggttctggaagaca

SEQ ID NO: 2 - RNA, 20 nt, synthetic construct
  source 1..20: mol_type = other RNA
ttggcccctcctcgcctggt

SEQ ID NO: 3 - RNA, 20 nt, synthetic construct
  source 1..20: mol_type = other RNA
ctgcagttggcccctcctcg

SEQ ID NO: 4 - RNA, 20 nt, synthetic construct
  source 1..20: mol_type = other RNA
tcaaacccctcacctgcagt

SEQ ID NO: 5 - RNA, 25 nt, synthetic construct
  source 1..25: mol_type = other RNA
cctgcagttggcccctcctcgcctg

SEQ ID NO: 6 - RNA, 25 nt, synthetic construct
  source 1..25: mol_type = other RNA
cccctcacctgcagttggcccctcc

SEQ ID NO: 7 - RNA, 17 nt, synthetic construct
  source 1..17: mol_type = other RNA
gcagttggcccctcctc

SEQ ID NO: 8 - RNA, 25 nt, synthetic construct
  source 1..25: mol_type = other RNA
cacctgcagttggcccctcctcgcc

SEQ ID NO: 9 - RNA, 25 nt, synthetic construct
  source 1..25: mol_type = other RNA
atcctcccacagagcctgtacggag

SEQ ID NO: 10 - RNA, 25 nt, synthetic construct
  source 1..25: mol_type = other RNA
ccctgatcctcccacagagcctgta

SEQ ID NO: 11 - RNA, 25 nt, synthetic construct
  source 1..25: mol_type = other RNA
ggcgttgccgcctttgccacaagat

SEQ ID NO: 12 - RNA, 25 nt, synthetic construct
  source 1..25: mol_type = other RNA
ctcacctctcctctcctcggcgttg

SEQ ID NO: 13 - RNA, 25 nt, synthetic construct
  source 1..25: mol_type = other RNA
ctccactcacctctcctctcctcgg

SEQ ID NO: 14 - RNA, 20 nt, synthetic construct
  source 1..20: mol_type = other RNA
agcagggcggctgtccccag

SEQ ID NO: 15 - RNA, 25 nt, synthetic construct
  source 1..25: mol_type = other RNA
gccacaagatgaccccaatgagcag

SEQ ID NO: 16 - RNA, 25 nt, synthetic construct
  source 1..25: mol_type = other RNA
cctttgccacaagatgaccccaatg

SEQ ID NO: 17 - RNA, 25 nt, synthetic construct
  source 1..25: mol_type = other RNA
tgccgcctttgccacaagatgaccc

SEQ ID NO: 18 - RNA, 25 nt, synthetic construct
  source 1..25: mol_type = other RNA
tcctcggcgttgccgcctttgccac

SEQ ID NO: 19 - RNA, 13 nt, synthetic construct
  source 1..13: mol_type = other RNA
tttgccacaagat

SEQ ID NO: 20 - RNA, 20 nt, synthetic construct
  source 1..20: mol_type = other RNA
ccgcctttgccacaagatga

SEQ ID NO: 21 - RNA, 25 nt, synthetic construct
  source 1..25: mol_type = other RNA
tttcttgttgaccatccccccagtc

SEQ ID NO: 22 - RNA, 25 nt, synthetic construct
  source 1..25: mol_type = other RNA
ccatccccccagtcacatgtgttgg

SEQ ID NO: 23 - RNA, 25 nt, synthetic construct
  source 1..25: mol_type = other RNA
cctcctcgcctggttctggaagaca

SEQ ID NO: 24 - RNA, 20 nt, synthetic construct
  source 1..20: mol_type = other RNA
ctggcccctcatcgccggtt

SEQ ID NO: 25 - RNA, 20 nt, synthetic construct
  source 1..20: mol_type = other RNA
cttcagctggcccctcatcg

SEQ ID NO: 26 - RNA, 20 nt, synthetic construct
  source 1..20: mol_type = other RNA
tccagtccctcaccttcagc

SEQ ID NO: 27 - RNA, 25 nt, synthetic construct
  source 1..25: mol_type = other RNA
tgggttgtcgttttcgccacaggat

SEQ ID NO: 28 - RNA, 23 nt, synthetic construct
  source 1..23: mol_type = other RNA
actactcccaggcctcccaggat

SEQ ID NO: 29 - RNA, 22 nt, synthetic construct
  source 1..22: mol_type = other RNA
gcagggctactactcccaggca

SEQ ID NO: 30 - RNA, 25 nt, synthetic construct
  source 1..25: mol_type = other RNA
tctcactcacctctcctcacgcctg

SEQ ID NO: 31 - RNA, 25 nt, synthetic construct
  source 1..25: mol_type = other RNA
cccagactcacccacagagcctgta

SEQ ID NO: 32 - RNA, 127 nt, Homo sapiens
  source 1..127: mol_type = mRNA
gctctgtgggaggatcagggctgggaactctagccctggccctggggatcctgggaggcctggggacagccgccctgctcattggggtcatcttgtggcaaaggcggcaacgccgaggagaggagag

SEQ ID NO: 33 - RNA, 17 nt, synthetic construct
  source 1..17: mol_type = other RNA
gcagttggcccctcctc

SEQ ID NO: 34 - RNA, 18 nt, Synthetic construct
  source 1..18: mol_type = other RNA
tcgttttcgccacaggat

(19) United States
(12) Patent Application Publication
(10) Pub. No.: US 2026/0276651 A1
(43) Pub. Date: Sep. 17, 2026

(54) METHODS OF MONITORING SPLICING

(71) Applicant: RAGE BIOTECH PTY LTD, Melbourne, Victoria (AU)

(72) Inventors: Merlin Christopher Thomas, Melbourne (AU); Raelene Jane Pickering, Melbourne (AU)

(73) Assignee: RAGE BIOTECH PTY LTD, Melbourne, Victoria (AU)

(21) Appl. No.: 19/480,294

(22) PCT Filed: May 3, 2024
(86) PCT No.: PCT/AU2024/050436

§ 371 Date: Oct. 30, 2025

Related U.S. Application Data
(63) Continuation-in-part of application No. PCT/AU2023/050959, filed on Oct. 5, 2023.

(30) Foreign Application Priority Data
May 5, 2023 (AU) 2023901363
Oct. 5, 2023 (WO) PCT/AU2023/050959

(51) Int. Cl. G01N 33/68

(52) U.S. Cl. CPC G01N 33/6884; G01N 2333/70503; G01N 2800/52

(57) ABSTRACT

The present invention relates to methods for identifying the splicing of RAGE pre-mRNA resulting in the inclusion of exon 9b and/or exclusion of exon 10 in a subject in a subject who has received, or is receiving an antisense oligonucleotide that modulates RAGE pre-mRNA splicing. In one aspect, the present invention provides a method for determining endogenous soluble receptor for advanced glycation end products (RAGE) level in the lung of a subject, the method comprising: determining the presence of endogenous soluble RAGE in a blood sample from a subject who has received or is receiving a treatment administered to their respiratory tract, wherein the treatment increases endogenous soluble RAGE, wherein the level of endogenous soluble RAGE in the blood sample correlates with the endogenous soluble RAGE level in the lung of the subject.

### RELATED APPLICATION

This application claims the benefit of priority from Australian provisional application no. 2023901363 filed 5 May 2023, and International Application no. PCT/AU2023/050959 filed 5 Oct. 2023, the entire disclosures of which is incorporated herein by reference.

### FIELD OF THE INVENTION

The present invention relates to methods for determining the level of Receptor for Advanced Glycation End-products (RAGE) isoforms, in particular endogenous soluble RAGE isoforms, or part thereof in a subject who is receiving, or has received, a treatment, such as an antisense oligonucleotide, that modulates RAGE pre-mRNA splicing.

### BACKGROUND OF THE INVENTION

The Receptor for Advanced Glycation End-products (RAGE) is a multivalent type I transmembrane glycoprotein belonging to the immunoglobulin (Ig) superfamily. The human RAGE (Ager) gene lies within the major histocompatibility complex class Ill region on chromosome 6. It comprises 11 exons and 10 introns, and a 5′ flanking region that regulates its transcription. The transcribed RAGE mRNA is ~1.4 kb, with a short 3′UTR.

The 50-55 kDa glycosylated RAGE protein is constitutively expressed in a limited range of cells (e.g. vascular endothelium, type I pneumocytes, leukocytes), although RAGE expression may be induced in most cell types and tissues following injury, stress, hypoxia or inflammation, providing a conduit for pro-inflammatory and pro-proliferative signalling. RAGE expression is consequently upregulated in inflammatory and metabolic disorders including but not limited to neurodegenerative disease, cancer, cardiovascular disease, diabetes, autoimmune and ischaemic injury in which RAGE is also implicated in the development and progression.

Under healthy conditions, the lungs' expression of RAGE is the highest of all tissues. Upregulation of RAGE signalling in the lung in other cells and at other sites has been implicated in a range of lung disorders including: chronic obstructive pulmonary disease (COPD)/emphysema; asthma; injury due to cigarette smoking/pollution; acute lung injury/Acute Respiratory Distress Syndrome; and pulmonary fibrosis.

The alternative splicing of RAGE is also important for the regulation of RAGE activity, through the generation of RAGE isoforms that have an altered ability to be activated by ligand-dependent and ligand-independent signalling pathways. The alternative splicing of RAGE is altered in disease states including malignancy, diabetes and Alzheimer's disease.

Antisense oligonucleotides (AONs) can be used to modulate alternative splicing of RAGE by targeting pre-mRNA RAGE. These AONs may be administered systemically or locally to modify the expression and/or activity of RAGE isoforms.

There is a need for a minimally invasive approach to determine the alternative splicing of RAGE pre-mRNA when AONs are administered to the respiratory tract of a subject.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a method for determining the level of endogenous soluble receptor for advanced glycation end products (RAGE) level in the lung of a subject, the method comprising:
determining the presence of endogenous soluble RAGE in a blood sample from a subject who has received or is receiving a treatment administered to their respiratory tract, wherein the treatment increases endogenous soluble RAGE,
wherein the level of endogenous soluble RAGE in the blood sample correlates with the endogenous soluble RAGE level in the lung of the subject.

In one aspect, the present invention provides a method for determining the presence of, or level of, endogenous soluble receptor for advanced glycation end products (RAGE) level in a blood sample of a subject, the method comprising:
determining the presence of, or level of, endogenous soluble RAGE in a blood sample from a subject who has received or is receiving a treatment administered to their respiratory tract, wherein the treatment increases endogenous soluble RAGE,
thereby determining the presence of, or level of, endogenous soluble RAGE in the blood sample of the subject.

In one aspect, the present invention provides a method for determining the splicing of Receptor for Advanced Glycation End-products (RAGE) pre-mRNA resulting in the inclusion of exon 9b and/or exclusion of exon 10 in a subject, the method comprising:
determining the presence of endogenous soluble RAGE in a blood sample from a subject who has received or is receiving an antisense oligonucleotide (AON), that promotes splicing in the RAGE pre-mRNA resulting in the inclusion of exon 9b and/or exclusion of exon 10, to the respiratory tract,
wherein the presence of endogenous soluble RAGE in the blood sample indicates the splicing of Receptor for Advanced Glycation End-products (RAGE) pre-mRNA resulting in the inclusion of exon 9b and/or exclusion of exon 10 in the subject.

In another aspect, the present invention provides a method for determining the splicing of Receptor for Advanced Glycation End-products (RAGE) pre-mRNA resulting in the inclusion of exon 9b and/or exclusion of exon 10 in a subject, the method comprising:
providing a blood sample from a subject who has received or is receiving an antisense oligonucleotide (AON) to the respiratory tract, wherein the AON promotes splicing in the RAGE pre-mRNA resulting in the inclusion of exon 9b and/or exclusion of exon 10
measuring the level of endogenous soluble RAGE in the blood sample,
wherein the level of endogenous soluble RAGE in the blood sample indicates the splicing of RAGE pre-mRNA resulting in the inclusion of exon 9b and/or exclusion of exon 10 in the subject.

In another aspect, the present invention provides a method for determining the splicing of Receptor for Advanced Glycation End-products (RAGE) pre-mRNA resulting in the inclusion of exon 9b and/or exclusion of exon 10 in a subject, the method comprising:
providing a blood sample from a subject who has received or is receiving an antisense oligonucleotide (AON), that promotes splicing in the RAGE pre-mRNA resulting in the inclusion of exon 9b and/or exclusion of exon 10, to the respiratory tract,
measuring the level of endogenous soluble RAGE in the blood sample,
wherein the level of endogenous soluble RAGE in the blood sample above a threshold level indicates the splicing of RAGE pre-mRNA resulting in the inclusion of exon 9b and/or exclusion of exon 10 in the subject.

In another aspect, the present invention provides a method for determining the splicing of Receptor for Advanced Glycation End-products (RAGE) pre-mRNA resulting in the inclusion of exon 9b and/or exclusion of exon 10 in a subject, the method comprising:
providing a blood sample from a subject prior to receiving an antisense oligonucleotide (AON), that promotes splicing in the RAGE pre-mRNA resulting in the inclusion of exon 9b and/or exclusion of exon 10, to the respiratory tract,
providing a blood sample from the subject after receiving an AON, that promotes splicing in the RAGE pre-mRNA resulting in the inclusion of exon 9b and/or exclusion of exon 10, to the respiratory tract,
measuring the level of endogenous soluble RAGE in each of the blood samples,
wherein a higher level of endogenous soluble RAGE in the blood sample from the subject after receiving the AON to the respiratory tract compared to the level of endogenous soluble RAGE in the blood sample from the subject prior to receiving the AON to the respiratory tract indicates splicing of RAGE pre-mRNA resulting in the inclusion of exon 9b and/or exclusion of exon 10 in the subject.

In another aspect, the present invention provides a method for determining the splicing of Receptor for Advanced Glycation End-products (RAGE) pre-mRNA resulting in the inclusion of exon 9b and/or exclusion of exon 10 in a subject, the method comprising:
obtaining a blood sample from a subject prior to receiving, to the respiratory tract, an antisense oligonucleotide (AON) that promotes splicing in the RAGE pre-mRNA resulting in the inclusion of exon 9b and/or exclusion of exon 10,
obtaining a blood sample from the subject after receiving, to the respiratory tract, an AON that promotes splicing in the RAGE pre-mRNA resulting in the inclusion of exon 9b and/or exclusion of exon 10,
measuring the level of endogenous soluble RAGE in each of the blood samples,
wherein a higher level of endogenous soluble RAGE in the blood sample from the subject after receiving the AON to the respiratory tract compared to the level of endogenous soluble RAGE in the blood sample from the subject prior to receiving the AON indicates splicing of RAGE pre-mRNA resulting in the inclusion of exon 9b and/or exclusion of exon 10 in the subject.

In another aspect, the present invention provides a method for determining whether a treatment/AON can increase endogenous soluble RAGE level in the respiratory tract, the method comprising:
administering a test treatment/AON to the respiratory tract of a subject,
obtaining a blood sample from the subject after the administration,
wherein the presence of, or an increase in the level of, endogenous soluble RAGE in the blood sample indicates the test treatment/AON increase endogenous soluble RAGE level in the respiratory tract.

In any aspect or embodiment herein, the blood sample comprises or consists of whole blood, plasma or serum.

In any aspect or embodiment, one or more or all steps of a method of the invention is performed in vitro or ex vivo.

In any aspect, the blood sample was obtained at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, or at least about 28 days after the subject received antisense oligonucleotide (AON) that promotes splicing in the RAGE pre-mRNA resulting in the inclusion of exon 9b and/or exclusion of exon 10 to the respiratory tract.

In any aspect, the blood sample was obtained at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, or at least 28 days after the subject received antisense oligonucleotide (AON) that promotes splicing in the RAGE pre-mRNA resulting in the inclusion of exon 9b and/or exclusion of exon 10 to the respiratory tract.

In any aspect, the blood sample was obtained 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, or 28 days after the subject received antisense oligonucleotide (AON) that promotes splicing in the RAGE pre-mRNA resulting in the inclusion of exon 9b and/or exclusion of exon 10 to the respiratory tract.

In any aspect, an AON may promote the production of endogenous soluble RAGE by promoting the inclusion of exon 9b and/or the exclusion (e.g. skipping) of exon 10. Therefore, in any aspect, the AON may promote splicing in the RAGE pre-mRNA resulting in the inclusion of exon 9b and/or skipping of exon 10. For example, the AON may result in an increase of the level of RAGE_v1, RAGE_v6, RAGE_v8, RAGE_v9, RAGE_v10, RAGE_v15, RAGE_v18, and RAGE_v19 mRNA, preferably an increase in the level of RAGE_v1, RAGE_v6, RAGE_v8, RAGE_v9, RAGE_v10, RAGE_v15, RAGE_v18, and RAGE_v19 mRNA in one or more tissues of the respiratory tract.

In any aspect, the endogenous soluble RAGE may be a polypeptide encoded by one or more of RAGE_v1, RAGE_v6, RAGE_v8, RAGE_v9 RAGE_v10, RAGE_v15, RAGE_v18, and RAGE_v19, preferably RAGE_v1 (ie esRAGE).

In any aspect, the AON is administered to the total respiratory tract, the upper respiratory tract or the lower respiratory tract.

As used herein, the upper respiratory tract may include any one or more of the following regions: the nose and nasal passages, paranasal sinuses, the pharynx, and the portion of the larynx above the vocal folds (cords). Typically, the lower respiratory tract includes any one or more of the following regions: the portion of the larynx below the vocal folds, trachea, bronchi and bronchioles. The lungs can be included in the lower respiratory tract and include the respiratory bronchioles, alveolar ducts, alveolar sacs, and alveoli.

In any aspect, the AON is administered as an aerosol, a dry powder or as nasal drops. In any embodiment, the AON may be administered using a nasal spray pump, intranasal installation, intratrachael instillation, metered dose inhaler (MDI), dry powder inhaler (DPI), nebuliser (jet, ultrasonic mesh or vibrating mesh) or soft mist inhaler (SMI).

In any aspect, the AON is an AON of 10 to 50 nucleotides comprising a targeting sequence complementary to a region near or within an intron of the RAGE pre-mRNA. Alternatively, the AON is 10 to 50 nucleotides comprising a targeting sequence complementary or adjacent to a splice site of the RAGE pre-mRNA.

Because factors such as RNA secondary structure, competition between AONs and SR proteins, heterogeneous nuclear ribonucleoproteins (hnRNPs), and/or other elements that make up the spliceosome can affect AON's' action, AONs directed at the crucial acceptor or donor splice sites will not always alter splicing. Consequently, in any aspect of the invention, the AON is 10 to 50 nucleotides comprising a targeting sequence complementary or adjacent to cis-acting RNA elements in the pre-mRNA of RAGE that act as enhancers or silencers, that, when bound by an elements of the splicosome (e.g. protein-splicing factors, uRNA, lncRNA) modulates the splicing of a nearby exon.

In any aspect, the AON is 10 to 50 nucleotides comprising a targeting sequence complementary to RAGE pre-mRNA which modulates secondary structure of said mRNA to influence splice site selection.

In any aspect, the AON is an isolated or purified AON for inducing exclusion (also known as skipping) of one or more exonic sequences in the RAGE gene transcript or part thereof.

In any aspect, the AON is an isolated or purified AON for inducing retention of intronic sequences in the RAGE gene transcript or part thereof.

In any aspect, the AON comprises at least one modified nucleotide. Typically, the AON is chemically-modified to prevent degradation of the pre-mRNA-AON complex, including but not limited to phosphorodiamidate morpholino oligomers (PMO), 2′ O-methyl phosphorothioate oligonucleotides (2OMe), and 2′-O-methoxyethyl phosphorothioate oligonucleotides (2MOE), locked nucleic acid (LNA) modified AONs, thermostable twisted intercalating nucleic acid (TINA) and peptide nucleic acids (PNAs).

In any aspect, the AON comprises at least one modified nucleotide selected from the group consisting of: phosphorodiamidate morpholino oligomers (PMO), 2′ O-methyl oligonucleotides (2OMe), 2′-O-methoxyethyl oligonucleotides (2′-MOE), phosphorothioate oligonucleotides, locked nucleic acid (LNA) modified AONs, thermostable twisted intercalating nucleic acid (TINA) and peptide nucleic acids (PNAs).

In any aspect, the AONs may be conjugated to moieties to increase their delivery, including but not limited to cell-penetrating peptides (CPPs), vivo-morpholinos (VMO) or peptide phosphorodiamidate morpholino oligomers (PPMO).

In any aspect, the antisense oligonucleotide comprises, consists essentially of or consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% complementary to a target region of exon 10 of RAGE pre-mRNA over the entirety of the antisense oligonucleotide. Preferably, the 5′-most nucleotide of the AON is nucleotide position 88 or 114 of exon 10 or is between nucleotide positions 88 to 114 of exon 10. Preferably, the 5′-most nucleotide of the target region is nucleotide position 88 or 114 of exon 10 or is between nucleotide positions 88 to 114 of exon 10.

In any embodiment, the 5′-most nucleotide of the target region is position 88 or 113, or between nucleotide positions 88 to 113 of exon 10 of RAGE pre-mRNA.

In any embodiment, the 5′-most nucleotide of the target region is position 90 or 113, or between nucleotide positions 90 to 113 of exon 10 of RAGE pre-mRNA.

In any embodiment, the 5′-most nucleotide of the target region is position 88 or 108, or between nucleotide positions 88 to 108 of exon 10 of RAGE pre-mRNA.

In any embodiment, the 5′-most nucleotide of the target region is position 90 or 108, or between nucleotide positions 90 to 108 of exon 10 of RAGE pre-mRNA.

In any embodiment, the 5′-most nucleotide of the target region is position 88 or 95, or between nucleotide positions 88 to 95 of exon 10 of RAGE pre-mRNA.

In any embodiment, the target region is from nucleotide position 88 to 137 of exon 10 of RAGE pre-mRNA.

In any embodiment, the target region is from nucleotide position 88 to 107 of exon 10 of RAGE pre-mRNA.

In any embodiment, the target region is from nucleotide position 90 to 102 of exon 10 of RAGE pre-mRNA.

In any embodiment, the target region is from nucleotide position 95 to 119 of exon 10 of RAGE pre-mRNA.

In any embodiment, the target region is between nucleotide positions 108 to 132 of exon 10 of RAGE pre-mRNA.

In any embodiment, the target region is between nucleotide positions 113 to 137 of exon 10 of RAGE pre-mRNA.

In any aspect, the AON may be 8 to 40 nucleotides in length, 15 to 25 nucleotides in length or 18 nucleotides in length.

In any embodiment, the AON is selected from the group comprising the sequences set forth in any of Tables 1a-1d. Preferably, the AON is selected from the list comprising: SEQ ID NO: 1-31, for example, the AON is SEQ ID NO: 11, 18, 19, or 20 or a nucleotide sequence at least 85%, 90% or 95% identical thereto.

The AONs for use in the methods of the present invention may be selected to be an AON capable of binding to a selected target site, wherein the target site is a putative mRNA splicing site selected from a splice donor site, splice acceptor site, splice enhancer sequences splice silencer sequences or sites that modulate the secondary structure of pre-mRNA. The target site may also include some flanking intronic sequences when the donor or acceptor splice sites are targeted.

More specifically, the AON may be selected from the group comprising of any one or more of SEQ ID NOs: 1-31 and/or the sequences set forth in any of Tables 1a-1d, and combinations or cocktails thereof. More preferably, the AON is SEQ ID NO: 11, 18, 19, or 20. The combination of AONs is preferably a combination of SEQ ID NO: 11 and 10, or SEQ ID NO: 11 and 13. This includes sequences which can hybridise to such sequences under stringent hybridisation conditions, sequences complementary thereto, sequences containing modified bases, modified backbones, and functional truncations or extensions thereof which possess or modulate pre-mRNA processing activity in a RAGE gene transcript.

In certain embodiments, AONs may be 100% complementary to the target sequence, or may include mismatches, e.g., to accommodate variants, as long as a hetero-duplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

The methods of the invention extends also to a combination of two or more AONs capable of binding to a selected target to modulate alternative splicing of the RAGE pre-mRNA, including a construct comprising two or more such AONs. The constructs may be used together for a combined AON-based therapy. The combination of AONs is preferably a combination of SEQ ID NO: 11 and 10, or SEQ ID NO: 11 and 13.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Division of the mouse lung as described in the Examples herein.

FIG. 2. Overview of experimental design. Time-dependent pharmacodynamics activity of a single intratracheal treatment with ASO m79 (3 mg/kg) in male C57BL/6 mice.

FIG. 3. esRAGE protein in the lung correlates with plasma concentration of esRAGE measured 14 days following single intratracheal treatment with ASO m79. Male C57BL/6 mice were administered a control oligo or ASO m79 (3 mg/kg) on day 0 and esRAGE levels were measured in the left lung tissue and plasma on day 14. Data shows mean±SEM; * p<0.05 vs control oligo.

FIG. 4. Overview of experimental design. Time-dependent pharmacodynamics activity of a single aerosolised dose with an 18-mer ASO (ASO2) (3 mg/kg or 0.3 mg/kg) in male C57BL/6 mice.

FIG. 5. esRAGE protein in the lung correlates with plasma concentration of esRAGE measured following single aerosolised treatment with ASO2. Male C57BL/6 mice were administered vehicle, ASO2 at 0.3 mg/kg or ASO2 at 3 mg/kg on day 0 and esRAGE levels were measured in the left lung tissue and plasma on day 10 (a) and day 14 (b). Both ASO2 at 0.3 mg/kg or ASO2 at 3 mg/kg levels showed a statistically significant increase in esRAGE levels compared to vehicle, Spearman Correlation p<0.05.

FIG. 6. Overview of experimental design. Dose-dependent pharmacodynamics activity of a single inhaled treatment with ASO2 (delivered by microsprayer) in male C57BL/6 mice.

FIG. 7. esRAGE protein in the lung correlates with plasma concentration of esRAGE measured 7 days following single inhaled treatment with ASO2. Male C57BL/6 mice were administered vehicle, ASO2 at 3 mg/kg or ASO2 at 10 mg/kg on day 0 and esRAGE levels were measured in the left lung tissue and plasma on day 7. Both ASO2 at 3 mg/kg or ASO2 at 10 mg/kg levels showed a statistically significant increase in esRAGE levels compared to vehicle, Spearman Correlation p<0.05.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

### RAGE Pre-mRNA Alternate Splicing

Alternative splicing is recognized as an important layer of post-transcriptional gene regulation for the Receptor for Advanced Glycation End-products (RAGE). Although most RAGE is expressed in its full length isoform, a number of different coding isoforms are generated through alternative splicing (also known as splicoforms), including splicoforms with N-terminal truncations, C-terminal truncations, and splicoforms retaining intronic sequences. These different splicoforms may act as possible regulators of the full-length RAGE receptor either by competitive ligand binding or by displacing the full-length protein from binding partners. Over twenty splicoforms have been identified in different tissues such as lung, liver, kidney, smooth muscle, endothelial cells and brain.

The different RAGE gene splice variants have been named RAGE, RAGE_v1 to RAGE_v19 according to the Human Gene Nomenclature Committee and are described in Hudson et al., (2008) The FASEB Journal, 22: 1572-1580, the contents of which are incorporated in its entirety.

As used herein, “endogenous soluble RAGE” refers to a polypeptide that lacks any signalling elements and/or the transmembrane domain of full-length RAGE. Endogenous soluble RAGE may be encoded, and subsequently translated, by an alternatively spliced RAGE pre-mRNA resulting in the inclusion of exon 9b and/or exclusion (eg skipping) of exon 10, resulting in a premature stop and the complete loss of the trans-membrane and cytoplasmic domains. For example, endogenous soluble RAGE may be encoded by one or more of RAGE_v1, RAGE_v6, RAGE_v8, RAGE_v9 RAGE_v10. RAGE_v15, RAGE_v18. and RAGE_v19 mRNA, preferably RAGE_v1. Therefore, it will be appreciated that any AON as described herein that promotes splicing in the RAGE pre-mRNA resulting in the inclusion of exon 9b and/or exclusion (eg skipping) of exon 10 may be used in the methods of the invention to increase “endogenous soluble RAGE” in the respiratory tract and plasma of a subject.

For example, an “endogenous soluble RAGE” as described herein may be endogenous secretory RAGE (esRAGE) which is encoded by RAGE_v1 mRNA. esRAGE constitutes ~5% of circulating RAGE in humans. The skipping of exon 10 in esRAGE-type splicing is ascribed to the limitation of intron length in higher eukaryotes. Roughly 45 nucleotides must separate the 5′ splice site and branch point, and the minimum distance between the branch point and 3′ splice site appears to be approximately 18 nucleotides, respectively. Therefore introns shorter than 70 nucleotides are extremely rare in mammals and cannot be spliced out efficiently. When the esRAGE 5′ splice site in intron 9 is selected, the distance between this site and the 3′ splice site that borders exon 10 is 46 nucleotides, which is considerably shorter than the lower limit of the intron length. Therefore, the use of the downstream, esRAGE 5′ splice site of intron 9 and the inclusion of exon 10 would be mutually exclusive. Among the known splice variants analyzed, all variants that used the downstream esRAGE 5′ splice site in intron 9 skipped exon 10; in contrast, all variants that used the upstream RAGE 5′ splice site in intron 9 included exon 10. Thus, the available evidence indicates that the selection of either one of the two alternative 5′ splice sites in intron 9 couples with inclusion or exclusion of exon 10. The means of regulation of this splicing or an external means to modulate has been previously unknown.

### Samples

In any aspect or embodiment herein, the blood sample comprises or consists of whole blood, plasma or serum.

A sample as used herein may be obtained from a subject or from components (e.g. cells) of a subject. The sample may be a “clinical sample” which is a sample derived from a patient. In one embodiment, the method of the invention is not practiced on a human or animal body, for example, the measurement of the level of endogenous soluble RAGE may be determined by analysing a previously obtained blood sample.

In one embodiment, the blood sample may be depleted of erythrocytes (e.g. in the form of plasma, serum or untreated whole blood). In one embodiment, erythrocytes are not present, or not present at significant levels, when endogenous soluble RAGE in the blood sample is measured. In another embodiment, erythrocytes are present at normal levels, i.e. they have not been depleted from the blood sample.

In any aspect, the blood sample was obtained at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, or at least about 28 days after the subject received antisense oligonucleotide (AON) that promotes splicing in the RAGE pre-mRNA resulting in the inclusion of exon 9b and/or exclusion of exon 10 to the respiratory tract.

In any aspect, the blood sample was obtained at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, or at least 28 days after the subject received antisense oligonucleotide (AON) that promotes splicing in the RAGE pre-mRNA resulting in the inclusion of exon 9b and/or exclusion of exon 10 to the respiratory tract.

In any aspect, the blood sample was obtained 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, or 28 days after the subject received antisense oligonucleotide (AON) that promotes splicing in the RAGE pre-mRNA resulting in the inclusion of exon 9b and/or exclusion of exon 10 to the respiratory tract.

In any aspect, the blood sample was obtained from about 7 days to about 28 days, from about 7 days to about 27 days, from about 7 days to about 26 days, from about 7 days to about 25 days, from about 7 days to about 24 days, from about 7 days to about 23 days, from about 7 days to about 22 days, from about 7 days to about 21 days, from about 7 days to about 20 days, from about 7 days to about 19 days, from about 7 days to about 18 days, from about 7 days to about 17 days, from about 7 days to about 16 days, from about 7 days to about 15 days or from about 7 days to about 14 days after the subject received antisense oligonucleotide (AON) that promotes splicing in the RAGE pre-mRNA resulting in the inclusion of exon 9b and/or exclusion of exon 10 to the respiratory tract.

In any aspect, the blood sample was obtained from 7 days to 28 days, from 7 days to 27 days, from 7 days to 26 days, from 7 days to 25 days, from 7 days to 24 days, from 7 days to 23 days, from 7 days to 22 days, from 7 days to 21 days, from 7 days to 20 days, from 7 days to 19 days, from 7 days to 18 days, from 7 days to 17 days, from 7 days to 16 days, from 7 days to 15 days or from 7 days to 14 days after the subject received antisense oligonucleotide (AON) that promotes splicing in the RAGE pre-mRNA resulting in the inclusion of exon 9b and/or exclusion of exon 10 to the respiratory tract.

Alternatively, in any aspect the blood sample was obtained no more than 28 days after the subject received antisense oligonucleotide (AON) that promotes splicing in the RAGE pre-mRNA resulting in the inclusion of exon 9b and/or exclusion of exon 10 to the respiratory tract. For example, the blood sample may be obtained less than 7 days, less than 14 days or no more than 28 days after the subject received antisense oligonucleotide (AON) that promotes splicing in the RAGE pre-mRNA resulting in the inclusion of exon 9b and/or exclusion of exon 10 to the respiratory tract.

In any aspect, the blood sample may be obtained and then frozen before being used for the methods of the present invention. For example, the blood sample may be obtained and frozen for an amount of time and under such storage conditions that would not substantially reduce the detectable levels of endogenous soluble RAGE as present in the blood sample before freezing.

In any embodiment, cord blood erythrocytes are depleted by ammonium chloride lysis, density gradient technique, hypotonic lysis, immunomagnetic cell separation or sedimentation, flow cytometric sorting, or equivalent methods appreciated by those skilled in the art.

### Detecting and Measuring Endogenous Soluble RAGE

The provided methods are based on the unexpected finding that the levels or presence of endogenous soluble RAGE in a blood sample is indicative of the splicing of Receptor for Advanced Glycation End-products (RAGE) pre-mRNA resulting in the inclusion of exon 9b and/or exclusion of exon 10 following administration of an AON that promotes splicing in the RAGE pre-mRNA resulting in the inclusion of exon 9b and/or exclusion of exon 10, to the respiratory tract. Thus, endogenous soluble RAGE levels in the blood can be used to monitor and determine the expression of alternate splicing of RAGE in the respiratory tract.

In any aspect, the present methods of the invention involve detecting the presence, or measuring the level, of endogenous soluble RAGE, as described herein, in a blood sample.

In any aspect, the level of endogenous soluble RAGE in a blood sample is higher or increased after receiving the AON to the respiratory tract compared to the level of endogenous soluble RAGE in the blood sample from the subject prior to receiving the AON.

The terms “presence” or “level” as used herein refers to an occurrence of, or a change in, a signal that is directly or indirectly detectable either by observation or by instrumentation. Typically, the detectable response is an occurrence of a signal wherein a fluorophore is inherently fluorescent. Alternatively, the detectable response is an optical response resulting in a change in the wavelength distribution patterns or intensity of absorbance or fluorescence or a change in light scatter, fluorescence lifetime, fluorescence polarization, or a combination of the above parameters. Other detectable responses include, for example, chemiluminescence, phosphorescence, radiation from radioisotopes, magnetic attraction, and electron density.

It is understood that the level or presence of endogenous soluble RAGE in a sample can be measured by any suitable method known in the art. Measurement of the expression level of endogenous soluble RAGE may be direct or indirect.

In one example, the level or presence of endogenous soluble RAGE may be measured using a labelled reagent, for example an antibody that specifically binds to endogenous soluble RAGE. The term “label,” as used herein, refers to a chemical moiety or protein that is directly or indirectly detectable (e.g. due to its spectral properties, conformation or activity), for example, when attached to an antibody.

Antibodies that specifically bind to endogenous soluble RAGE, such as esRAGE, are known in the art and can be purchased commercially. For example, esRAGE antibodies can be purchased from Sigma-Aldrich, Australia (Product number: MAB5328).

A detection label conjugated to a reagent may be any label that allows separate detection and quantitation, by flow cytometry. For example, a fluorochrome. Suitable fluorescent labels are known in the art and include fluorescein isothiocyanate (FITC), phycoerythrin (PE), peridin chlorophyll protein (PerCP), allophycocyanin (APC), Alexa fluor 488, Alexa fluor 647, Alexa fluor 710, Alexa fluor 405, cyanin 5 (Cy5), Cyanin 5.5 (Cy5.5), pacific blue (PacB), horizon violet 450 (HV450), pacific orange (PacO), horizon-V500 (HV500), Krome Orange, Brilliant Violet 421 (BV421), Brilliant Violet 510 (BV510), Brilliant Violet 605 (BV605), Brilliant Violet 650 (BV650), Brilliant Violet 711 (BV711), Brilliant Violet 785 (BV785), Brilliant Ultraviolet 395 (BUV395), Brilliant Ultraviolet 496 (BUV496), Brilliant Ultraviolet 737 (BUV737), Orange Cytognos (OC)515, quantum dots and conjugates thereof coupled with PE, to APC or to PerCP (e.g. PE/Cy5, PE/Cy5.5, PE/Cy7, PerCP/Cy5.5, APC/Cy7, APC-H7, APC-Alex750, PE-Texas Red, PE-Dazzle, PE-CF594) or any additional compatible fluorochrome or fluorochrome tandem, etc.

In one example, antibodies are conjugated to (1) pacific blue (PacB), brilliant violet 421 (BV421) or Horizon V450; (2) pacific orange (PacO), Horizon V500 (HV500), BV510, Khrome orange (KO) or OC515, (3) Horizon BB515, fluorescein isothiocyanate (FITC) or Alexa488, (4) phycoerythrin (PE), (5) peridinin chlorophyl protein/cyanine 5.5 (PerCP-Cy5.5), PerCP or PE-TexasRed, (6) phycoerythrin/cyanine7 (PE-Cy7), (7) allophycocyanine (APC) or Alexa647, and (8) allophycocyanine/hilite 7 (APC-H7), APC-Cy7, Alexa680, APC-A750, APC-C750 or Alexa700.

In another example, the antibodies are conjugated to (1) brilliant violet 421, (2) brilliant violet 510 (BV510), (3) brilliant violet 650 (BV650), (4) brilliant violet 786 (BV786), (5) fluorescein isothiocyanate (FITC), (6) peridinin chlorophyl protein/cyanine 5.5 (PerCP-Cy5.5), (7) to phycoerythrin (PE), (8) phycoerythrin/cyanine7 (PE-Cy7), (9) allophycocyanine (APC), and (10) allophycocyanine/H7 (APC-H7), APC-C750 or APC-Alexa750.

A suitable label may be directly or indirectly linked to a reagent via the use of a suitable tag. In a preferred embodiment, the detectable label is linked to streptavidin.

The method of the present invention further comprises contacting the blood sample with a reagent that allows endogenous soluble RAGE to be identified. Typically, molecule is bound or is itself the detectable label. For example, the molecule may be a fluorescent dye, antibody, or enzyme that leads to substrate being produced. Alternatively, the reagent is linked to a tag that facilitates binding to the detectable label. For example, the tag may bind non-covalently to, or form a covalent interaction with, the detectable label. Suitable tags are known in the art and have been described herein.

For example, the reagent may be an antibody that detects endogenous soluble RAGE and the detectable label is a fluorochrome. Suitable fluorochromes are known in the art and have been described herein.

Preferably, the reagent is an antibody that specifically binds to the c-terminal domain of endogenous soluble RAGE as defined within but does not significantly bind other RAGE isoforms. Due to the unique truncation of the transmembrane and intracellular signalling domains, the endogenous soluble RAGE proteins as described herein, have unique conformational epitopes that are not found on other RAGE isoforms which may be targeted to provide specific detection/measurement of endogenous soluble RAGE levels. Such antibodies and kits for detecting endogenous soluble RAGE (e.g. esRAGE) can be commercially purchased, for example Human esRAGE ELISA kit (HUF104782; Assay Genie).

The detection of reagents linked to detectable labels binding to endogenous soluble RAGE may be performed by biochemical techniques such antibody binding, enzyme linked immunosorbent assay (ELISA), Western blotting, radioimmunoassay (RIA), other immunoassays, and fluorescence activated cell analysis (FACS). These methods are practiced as known in the art. ELISA kits suitable for detecting endogenous soluble RAGE, such as esRAGE, can be obtained commercially, for example, from mybiosource.com (catalogue #MBS015358).

Where the sample is bound to a fluorochrome selective reagent is used, flow cytometry or microscopy may be used to detect the presence or level of endogenous soluble RAGE labelled with the reagent. Such methods are practiced as known in the art.

In any aspect, the level of endogenous soluble RAGE (e.g esRAGE) measured in a blood sample from a subject who has received or is receiving an AON as described herein of at least about 1.1-, 1.2-, 1.5-, 2-, 3-, 4-, 5-, 7-, 10-, 20-, 100-fold and higher values compared the level of endogenous soluble RAGE measured in a blood sample from the same subject before receiving the AON.

The level of endogenous soluble RAGE in a blood sample from a subject may refer to the concentration, amount, level, relative concentration, relative amount or activity of endogenous soluble RAGE. Generally, the level endogenous soluble RAGE is indicative of splicing of RAGE pre-mRNA resulting in the inclusion of exon 9b and/or exclusion of exon 10 in the subject, preferably within cells of the respiratory tract.

In any aspect, measuring the level of, or presence of, endogenous soluble RAGE comprises performing an in vitro assay. In any embodiment, the in vitro assay is an immunoassay, an aptamer-based assay, or a histological or cytological assay. In some instances, the values of the one or more biomarkers are measured by an enzyme-linked immunosorbent assay (ELISA), immunoblotting, immunoprecipitation, radioimmunoassay (RIA), immunostaining, a flow cytometry assay, surface plasmon resonance (SPR), a chemiluminescence assay, a lateral flow immunoassay, an inhibition assay or an avidity assay.

In one technique to compare protein levels of expression from two different samples each sample is separately subjected to 2D gel electrophoresis. Alternatively, each sample is differently labeled and both samples are loaded onto the same 2D gel. See, e.g., Unlu et al. Electrophoresis, 1997; 18:2071-2077, which is incorporated by reference herein for at least its teachings of methods to assess and compare levels of protein expression. The same protein in each sample is identified by the relative position within the pattern of proteins resolved by 2D electrophoresis. The expression levels of the protein in a first sample is then compared to the expression level of the same protein in the second sample, thereby allowing the identification of a protein that is expressed differently between the two samples (e.g., a biomarker). This comparison is preferably made for subjects before and after they receive an AON that promotes splicing in the RAGE pre-mRNA resulting in the inclusion of exon 9b and/or exclusion of exon 10.

In another technique, the expression level of endogenous soluble RAGE is in a single sample as a percentage of total expressed proteins. This assessed level of expression is compared to a preexisting reference standard, thereby allowing for the identification of endogenous soluble RAGE that is differentially expressed in the sample relative to the reference standard.

In any aspect, increased endogenous soluble RAGE may involve comparison with a reference standard. A reference standard may be from one or more subjects who (a) may be, or may not be, suffering from a RAGE-related disease or disease and/or (b) have not been administered a RAGE splicing AON as described herein. Therefore, if the levels of expression of endogenous soluble RAGE in the blood sample from the subject who has received or is receiving an AON is increased or higher compared to the levels of expression of endogenous soluble RAGE in the reference standard, preferably a statistically significant increase, then a determination is made that the subject has an increase in splicing of RAGE pre-mRNA resulting in the inclusion of exon 9b and/or exclusion of exon 10, preferably in the cells of the respiratory tract. In some embodiments a statistically significant increase is equal to or greater than 2 standard deviations above the reference standard (an example value for the reference standard is described below). Alternatively, if the levels of expression of endogenous soluble RAGE in the sample from the subject who has received or is receiving an AON is the same or not statistically significant compared to the levels of expression of endogenous soluble RAGE in the reference standard, then a determination is made that the subject does not have an increase in splicing of RAGE pre-mRNA resulting in the inclusion of exon 9b and/or exclusion of exon 10, preferably in the cells of the respiratory tract. Therefore, in any method or use of the invention, where a determination is made that the subject does not have an increase in splicing of RAGE pre-mRNA resulting in the inclusion of exon 9b and/or exclusion of exon 10, the method or use further comprises the step of further administering another dose of the AON as described herein.

As used herein, a threshold level of a endogenous soluble RAGE may be a level determined from one or more control subjects who have not received a treatment that increases endogenous soluble RAGE. In one embodiment, those control subjects may be healthy or otherwise normal subjects that do not have a RAGE mediated condition or respiratory condition or disease as defined herein and who have not received a treatment that increases endogenous soluble RAGE. In another embodiment, those control subjects may have a RAGE mediated condition or respiratory condition or disease as defined herein and who have not received a treatment that increases endogenous soluble RAGE. An exemplary threshold level, or reference standard, may be 250±25 pg/mL (mean±SD).

Endogenous soluble RAGE data may be analyzed by a variety of methods to identify endogenous soluble RAGE and determine the statistical significance of differences in observed levels of endogenous soluble RAGE between test and reference expression profiles, for example in blood samples taken before and after receiving an AON as described herein, in order to indicate the AON treatment has resulted in splicing of RAGE pre-mRNA resulting in the inclusion of exon 9b and/or exclusion of exon 10 in the subject.

### Antisense Oligonucleotides (AONs)

Antisense oligonucleotide (AON) are short, synthetic, antisense, modified strands of DNA or RNA that can selectively hybridise to pre-RNA/mRNA through Watson-Crick base pairing and selectively modulate the function of the target RNA.

The terms “AON” and “ASO” are both abbreviations of the term “antisense oligonucleotide” and are used interchangeably herein.

When AONs are used to modulate alternative splicing of mRNA, they are often referred to as splice-switching oligonucleotides (SSO). In the present invention, the terms AON and SSO may be used interchangeably. SSOs base-pair with a pre-mRNA and disrupt the normal splicing repertoire of the transcript by blocking the RNA-RNA base-pairing or protein-RNA binding interactions that occur between components of the splicing machinery and the pre-mRNA. SSOs can induce “skipping” of selected exons and/or retention of intronic sequences to modulate the product of translation. This can be achieved by targeting splice sites directly or by targeting cis-acting sequences involved in enhancing or silencing splicing by modulating binding of specific proteins or altering secondary structure of the pre mRNA.

Therapeutic SSOs may be used for the treatment of genetic disorders, to skip faulty or misaligned sections allowing for the generation of internally deleted, but now functional protein as a therapy.

The AONs for use in the methods of the present invention selectively manipulate the alternative splicing pattern of RAGE pre-mRNA, resulting in the generation of either natural RAGE mRNA splicoforms that are either non-functional or that act as a decoy receptor to antagonise ligand dependent activation and ligand-independent transactivation of full length RAGE.

Notably, there are no common RAGE polymorphisms at these splice sites. The RAGE sequence is highly conserved. Therefore personalisation or subjectised sequence modification is not required, unlike the management of genetic disorders with exon skipping technologies.

According to any aspect of the methods of the invention, the AONs as described herein are capable of binding to a selected target on a RAGE gene transcript to modulate pre-mRNA splicing in a RAGE gene transcript or part thereof. Broadly, the AON may be an isolated or purified AON. By “isolated” is meant material that is substantially or essentially free from components that normally accompany it in its native state. For example, an “isolated polynucleotide” or “isolated oligonucleotide,” as used herein, may refer to a polynucleotide that has been purified or removed from the sequences that flank it in a naturally-occurring state, e.g., a DNA fragment that is removed from the sequences that are adjacent to the fragment in the genome. The term “isolating” as it relates to cells, refers to the purification of cells (e.g., fibroblasts, lymphoblasts) from a source subject (e.g., a subject with a polynucleotide repeat disease). In the context of DNA, mRNA or protein, “isolating” refers to the recovery of the DNA, mRNA or protein from a source, e.g., cells.

According to any aspect of the methods of the invention, the AONs as described herein promote splicing in the RAGE pre-mRNA resulting in the inclusion of exon 9b and/or exclusion (eg skipping) of exon 10, resulting in a premature stop and the complete loss of the trans-membrane and cytoplasmic domains. Therefore, the AONs as described herein may increase the level of RAGE_v1, RAGE_v6, RAGE_v8, RAGE_v9, RAGE_v10, RAGE_v15, RAGE_v18, and RAGE_v19 mRNA, preferably an increase in the level of RAGE_v1, RAGE_v6, RAGE_v8, RAGE_v9, RAGE_v10, RAGE_v5, RAGE_v18, and RAGE_v19 mRNA in a tissue or sample. For example, an AON as described herein may increase RAGE_v1 mRNA levels in a tissue or sample by promoting splicing in the RAGE pre-mRNA that results in the skipping of exon 10 and retention of intron 9 (exon 9b). In another example, an AON as described herein may increase RAGE_v10 mRNA levels in a tissue or sample by promoting splicing in the RAGE pre-mRNA that results in the skipping of exon 10 and exon 11.

An AON can be said to be “directed to” or “targeted against” a target sequence with which it hybridizes. In certain embodiments, the target sequence includes a region including a 3′ or 5′ splice site of a pre-processed mRNA, a branch point, or other sequences involved in the regulation of splicing, including splice enhancers and splice silencers and sites determining the secondary structure of RNA that influence splicing. The target sequence may be within an exon or within an intron or spanning an intron/exon junction.

In any aspect, the antisense oligonucleotide comprises, consists essentially of or consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% complementary to a target region of exon 10 of RAGE pre-mRNA over the entirety of the antisense oligonucleotide. Preferably, the 5′-most nucleotide of the AON is nucleotide position 88 or 114 of exon 10 or is between nucleotide positions 88 to 114 of exon 10. Preferably, the 5′-most nucleotide of the target region is nucleotide position 88 or 114 of exon 10 or is between nucleotide positions 88 to 114 of exon 10. Typically, the nucleotide position may be identical or equivalent to SEQ ID NO: 32, where the first nucleotide is position 1.

| | |
|---|---|
| Human RAGE | GCTCTGTGGGAGGATCAGGGCTGGGAACT |
| Exon 10 | CTAGCCCTGGCCCTGGGGATCCTGGGAGG |
| (SEQ ID NO: 32) | CCTGGGGACAGCCGCCCTGCTCATTGGGG |
| | TCATCTTGTGGCAAAGGCGGCAACGCCGA |
| | GGAGAGGAGAG |

In certain embodiments, the AON has sufficient sequence complementarity to a target RNA (i.e., the RNA for which splice site selection is modulated) to block a region of a target RNA (e.g., pre-mRNA) in an effective manner. In exemplary embodiments, such blocking of RAGE pre-mRNA serves to modulate splicing, either by masking a binding site for a splicosomal protein that would otherwise modulate splicing and/or by altering the structure of the targeted RNA. In some embodiments, the target RNA is target pre-mRNA (e.g., RAGE gene pre-mRNA).

An AON having a sufficient sequence complementarity to a target RNA sequence to modulate splicing of the target RNA means that the AON has a sequence sufficient to trigger the masking of a binding site for a native protein that would otherwise modulate splicing and/or alters the three-dimensional structure of the targeted RNA.

Selected AONs can be made shorter, e.g., about 12 bases, or longer, e.g., about 50 bases, and include a small number of mismatches, as long as the sequence is sufficiently complementary to effect splice modulation upon hybridization to the target sequence, and optionally forms with the RNA a heteroduplex having a Tm of 45° C. or greater.

Preferably, the AON is selected from the group comprising SEQ ID NOS: 1-31 and/or the sequences set forth in any of Tables 1a-1d. More preferably, the AON is SEQ ID NO: 11, 18, 19, or 20.

In certain embodiments, the degree of complementarity between the target sequence and AON is sufficient to form a stable duplex. The region of complementarity of the AONs with the target RNA sequence may be as short as 8-11 bases, but can be 12-15 bases or more, e.g., 10-50 bases, 10-40 bases, 12-30 bases, 12-25 bases, 15-25 bases, 12-20 bases, or 15-20 bases, including all integers in between these ranges. An AON of about 16-17 bases is generally long enough to have a unique complementary sequence. In certain embodiments, a minimum length of complementary bases may be required to achieve the requisite binding Tm, as discussed herein.

In certain embodiments, oligonucleotides as long as 50 bases may be suitable, where at least a minimum number of bases, e.g., 10-12 bases, are complementary to the target sequence. In general, however, facilitated or active uptake in cells is optimized at oligonucleotide lengths of less than about 30 bases. For phosphorodiamidate morpholino oligomer (PMO) AONs described further herein, an optimum balance of binding stability and uptake generally occurs at lengths of 18-25 bases. Included are AONs (e.g., PMOs, PMO-X, PNAs, LNAs, TINA, 2′-OMe) that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases.

In certain embodiments, AONs may be 100% complementary to the target sequence, or may include mismatches, e.g., to accommodate variants, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability. Although such an AON is not necessarily 100% complementary to the target sequence, it is effective to stably and specifically bind to the target sequence, such that splicing of the target pre-RNA is modulated.

The stability of the duplex formed between an AON and a target sequence is a function of the binding Tm and the susceptibility of the duplex to cellular enzymatic cleavage. The Tm of an oligonucleotide with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligonucleotide Hybridization Techniques, Methods Enzymol. Vol. 154 pp. 94-107. In certain embodiments, AONs may have a binding Tm, with respect to a complementary-sequence RNA, of greater than body temperature and preferably greater than about 45° C. or 50° C. Tm's in the range 60-80° C. or greater are also included.

Additional examples of variants include AONs having about or at least about 70% sequence identity or homology, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or homology, over the entire length of any of SEQ ID NOS: 1-31 and/or the sequences set forth in any of Tables 1a-1d. More preferably, the AON is SEQ ID NO: 11, 18, 19, or 20.

The modification of pre-mRNA splicing preferably induces “skipping”, or the removal of one or more exons or retention of introns of the mRNA. The resultant protein is preferably of a shorter length when compared to the parent full-length RAGE protein due to either internal truncation or premature termination. Preferably, the resultant protein has a C-terminal truncation. These truncated RAGE proteins may be termed splicoforms of the full length RAGE protein.

The remaining exons of the mRNA generated may be in-frame and produce a shorter protein with a sequence that is similar to that of the parent full length protein, except that it has an internal truncation in a region between the original 3′ and 5′ ends. In another possibility, the exon skipping may induce a frame shift that results in a protein wherein the first part of the protein is substantially identical to the parent full length protein, but wherein the second part of the protein has a different sequence (e.g. a nonsense sequence) due to a frame-shift. Alternatively, the exon skipping may induce the production of a prematurely terminated protein due to a disruption of the reading frame and presence of a premature termination of translation. The prematurely terminated protein may be the result of mRNA that is prematurely terminated (e.g. skipping of exons 10 and/or 11) or may be the result of a run on into an intron (e.g. RAGE 9b) or missense skip which provides an mRNA that contains the exon 10 and/or 11 mRNA, but which does not provide expression of the protein encoded by these exons.

Skipping subject exons of exons 1 to 9 will preferably disrupt the reading frame of the RAGE transcript. This will lead to increased degradation of RNA through nonsense mediated decay.

Skipping subject exons of exons 1 to 11 will preferably keep the reading frame intact. This will preferably lead to translation into an internally truncated protein. The truncated protein or RAGE mRNA splicoform may have a completely ablated function, may have a reduced function or act as a decoy receptor.

Preferably, these truncated, nonsense or prematurely terminated proteins are lacking one or more functional domains involved the induction of intracellular signalling pathways by RAGE ligands or non-ligand-dependent transactivation of RAGE by collocated GPCRs. For example, Exon 10 encodes a transmembrane domain and removing this exon may generate a soluble RAGE protein, which could potentially act as a soluble decoy or competitive antagonist of ligand induced signalling via RAGE. Truncated, nonsense or prematurely terminated proteins may further lack an attachment or binding site for other factors, removal of which may lead to a reduction in interaction of the RAGE protein with relevant signalling pathways.

Alternatively, the removal of one or more exons may lead to misfolding of the RAGE protein and a reduction in the ability of the protein to be successfully transported through the membrane.

The presence of internally truncated proteins (i.e. proteins lacking the amino acids encoded by one or more exons) is preferable. If the RAGE protein is inhibited, there may be problems with elevation of RAGE transcription as the body tries to compensate for the reduction in the total amount of RAGE protein. In contrast, the presence of an internally truncated protein (preferably lacking one or more of the features of the complete RAGE protein), should be sufficient to prevent elevated transcription, but still provide a therapeutic advantage due to a reduction in the total amount of functional RAGE protein.

The AON induced exon skipping as described herein need not completely or even substantially ablate the function of the RAGE protein. Preferably, the modulation of alternative splicing via the exon skipping process results in a reduced or compromised functionality of the RAGE protein.

The different isoforms of RAGE produced using different skipping strategies could result in proteins with ablated or reduced signalling activity that could preferably be used to treat or prevent different diseases associated with RAGE activity, such as neurodegenerative diseases, cancer, lung disorders, or inflammatory diseases. Alternative splicing strategies may form truncated proteins or proteins with reduced functions that can be preferably used as treatments for specific aspects, forms or progression of the diseases associated with RAGE expression and activity.

The skipping process using AONs may exclude (skip) a subject exon, or may result in skipping two or more exons at once.

The skipping process using AONs may include retention of intronic sequences with or without directly skipping one or more exons.

The AONs for use in the methods of the present invention may be a combination of two or more AONs capable of binding to a selected target to induce exon exclusion in a RAGE gene transcript. The combination may be a cocktail of two or more AONs and/or a construct comprising two or more or two or more AONs joined together.

TABLE 1a
Sequence of AONs for modulation of alternative splicing in human RAGE
Exon 9

| SEQ ID NO | AON# | Name | Sequence (5′ to 3′) |
|---|---|---|---|
| 1 | 3668 | H-AGERex9A(−10+15) | CCUCCUCGCCUGGUUCUGGAAGACA |
| 2 | 3669 | H-AGERex9A(+02+21) | UUGGCCCCUCCUCGCCUGGU |
| 3 | 3670 | H-AGERex9A(+08+27) | CUGCAGUUGGCCCCUCCUCG |
| 4 | 3671 | H-AGERex9D(+07−13) | UCAAACCCCUCACCUGCAGU |
| 5 | 4103 | H-AGERex9A(+3+27) | CCUGCAGUUGGCCCCUCCUCGCCUG |
| 6 | 4104 | H-AGERex9A(+10+27) | CCCCUCACCUGCAGUUGGCCCCUCC |
| 7 | 4105 | H-AGERex9A(+8+25) | GCAGUUGGCCCCUCCUC |
| 8 | 4106 | H-AGERex9D(+22−3) | CACCUGCAG UUG GCCCCUCCUCGCC |

TABLE 1b
Sequence of AONs for modulation of alternative splicing in human RAGE
Exon 10

| SEQ ID NO | AON# | Name | Sequence (5′ to 3′) |
|---|---|---|---|
| 9 | 3777 | AGER_H10A(−10+15) | AUCCUCCCACAGAGCCUGUACGGAG |
| 10 | 3778 | AGER_H10A(−5+20) | CCCUGAUCCUCCCACAGAGCCUGUA |
| 11 | 3779 | AGER_H10A(+90+114) | GGCGUUGCCGCCUUUGCCACAAGAU |
| 12 | 3780 | AGER_H10D(+20−5) | CUCACCUCUCCUCUCCUCGGCGUUG |
| 13 | 3781 | AGER_H10D(+15−10) | CUCCACUCACCUCUCCUCUCCUCGG |
| 14 | 82 | AGER_H10A(+60+79) | AGCAGGGCGGCUGUCCCCAG |
| 15 | 83 | AGER_H10A(+75+99) | GCCACAAGAUGACCCCAAUGAGCAG |
| 16 | 84 | AGER_A10(+80+104) | CCUUUGCCACAAGAUGACCCCAAUG |
| 17 | 85 | AGER_A10(+85+109) | UGCCGCCUUUGCCACAAGAUGACCC |
| 18 | 87 | AGER_H10(+95+119) | UCCUCGGCGUUGCCGCCUUUGCCAC |
| 19 | 90 | AGER_H10A(+90+102) | UUUGCCACAAGAU |
| 20 | 93 | AGER_H10A(+88+107) | CCGCCUUUGCCACAAGAUGA |

TABLE 1c
Sequence of AONs for modulation of alternative splicing in human RAGE
Intron 9

| SEQ ID NO | AON# | Name | Sequence (5′ to 3′) |
|---|---|---|---|
| 21 | 88 | AGER_H10A(−75−51) | UUUCUUGUUGACCAUCCCCCCAGUC |
| 22 | 89 | AGER_H10A(−86−62) | CCAUCCCCCCAGUCACAUGUGUUGG |

TABLE 1d
Sequence of AONs for modulation of alternative splicing in murine RAGE

| SEQ ID NO | AON# | Name | Sequence (5′ to 3′) |
|---|---|---|---|
| 23 | 3672 | M-AgerEx9A(−10+15) | CCUCCUCGCCUGGUUCUGGAAGACA |
| 24 | 3673 | M-AgerEx9A(+02+21) | CUGGCCCCUCAUCGCCGGUU |
| 25 | 3674 | M-AgerEx9A(+08+27) | CUUCAGCUGGCCCCUCAUCG |
| 26 | 3675 | M-AgerEx9D(+07−13) | UCCAGUCCCUCACCUUCAGC |
| 27 | m3779 | M-AgerEx10A(+90+114) | UGGGUUGUCGUUUUCGCCACAGGAU |
| | (m79) | | |
| 28 | m97 | M-AgerEx10A(+48+70) | ACUACUCCCAGGCCUCCCAGGAU |
| 29 | m98 | M-AgerEx10A(+56+79) | GCAGGGCUACUACUCCCAGGCA |
| 30 | m101 | M-AgerEx10D(−15+10) | UCUCACUCACCUCUCCUCACGCCUG |
| 31 | m102 | M-AgerEx10A(−5+20) | CCCAGACUCACCCACAGAGCCUGUA |
| 34 | ASO2 | M-AgerEx10A(+90+107) | TCGTTTTCGCCACAGGAT |

More specifically, the AON for use in the methods of the present invention may be selected from those set forth in any of Tables 1a-1d. The sequences are preferably selected from the group consisting of any one or more of any one or more of SEQ ID NOs: 1-31, and combinations or cocktails thereof. More preferably, the AON is SEQ ID NO: 11, 18, 19, or 20. The combination of AONs is preferably a combination of SEQ ID NO: 11 and 10, or SEQ ID NO: 11 and 13. This includes sequences which can hybridise to such sequences under stringent hybridisation conditions, sequences complementary thereto, sequences containing modified bases, modified backbones, and functional truncations or extensions thereof which possess or modulate pre-mRNA processing activity in a RAGE gene transcript.

The oligomer and the DNA, cDNA or RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or pairing such that stable and specific binding occurs between the oligomer and the DNA, cDNA or RNA target. It is understood in the art that the sequence of an AON need not be 100% complementary to that of its target sequence to be specifically hybridisable. An AON is specifically hybridisable when binding of the compound to the target DNA or RNA molecule interferes with the normal function of the target DNA or RNA product, and there is a sufficient degree of complementarity to avoid non-specific binding of the AON to non-target sequences under conditions in which specific binding is desired, i.e., under physiological conditions in the case of in vivo assays or therapeutic treatment, and in the case of in vitro assays, under conditions in which the assays are performed.

Selective hybridisation may be under low, moderate or high stringency conditions, but is preferably under high stringency. Those skilled in the art will recognise that the stringency of hybridisation will be affected by such conditions as salt concentration, temperature, or organic solvents, in addition to the base composition, length of the complementary strands and the number of nucleotide base mismatches between the hybridising nucleic acids. Stringent temperature conditions will generally include temperatures in excess of 30° C., typically in excess of 37° C., and preferably in excess of 45° C., preferably at least 50° C., and typically 60° C.-80° C. or higher. Stringent salt conditions will ordinarily be less than 1000 mM, typically less than 500 mM, and preferably less than 200 mM. However, the combination of parameters is much more important than the measure of any single parameter. An example of stringent hybridisation conditions is 65° C. and 0.1×SSC (1×SSC=0.15 M NaCl, 0.015 M sodium citrate pH 7.0). Thus, the AONs for use in the methods of the present invention may include oligomers that selectively hybridise to the sequences provided in any of Tables 1a-1d, or SEQ ID NOs:1-31. More preferably, the AON is SEQ ID NO: 11, 18, 19, or 20.

It will be appreciated that the codon arrangements at the end of exons in structural proteins may not always break at the end of a codon, consequently there may be a need to delete more than one exon from the pre-mRNA to ensure in-frame reading of the mRNA. In such circumstances, a plurality of AONs may need to be selected by the method of the invention wherein each is directed to a different region responsible for inducing inclusion of the desired exon and/or intron. At a given ionic strength and pH, the Tm is the temperature at which 50% of a target sequence hybridizes to a complementary polynucleotide. Such hybridization may occur with “near” or “substantial” complementarity of the AON to the target sequence, as well as with exact complementarity.

Typically, selective hybridisation will occur when there is at least about 55% identity over a stretch of at least about 14 nucleotides, preferably at least about 65%, more preferably at least about 75% and most preferably at least about 90%, 95%, 98% or 99% identity with the nucleotides of the AON. The length of homology comparison, as described, may be over longer stretches and in certain embodiments will often be over a stretch of at least about nine nucleotides, usually at least about 12 nucleotides, more usually at least about 20, often at least about 21, 22, 23 or 24 nucleotides, at least about 25, 26, 27 or 28 nucleotides, at least about 29, 30, 31 or 32 nucleotides, at least about 36 or more nucleotides.

Thus, the AON for use in the methods of the present invention sequences preferably have at least 75%, more preferably at least 85%, more preferably at least 86, 87, 88, 89 or 90% homology to the sequences shown in the sequence listings herein. More preferably there is at least 91, 92, 93 94, or 95%, more preferably at least 96, 97, 98% or 99%, homology. Generally, the shorter the length of the AON, the greater the homology required to obtain selective hybridisation. Consequently, where an AON consists of less than about 30 nucleotides, it is preferred that the percentage identity is greater than 75%, preferably greater than 85, 86, 87, 88, 89, 90, 91, 92, 93, 94, 95%, 96, 97, 98% or 99% compared with the AONs set out in the sequence listings herein. Nucleotide homology comparisons may be conducted by sequence comparison programs such as the GCG Wisconsin Bestfit program or GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

The AONs for use in the methods of the present invention may have regions of reduced homology, and regions of exact homology with the target sequence. It is not necessary for an oligomer to have exact homology for its entire length. For example, the oligomer may have continuous stretches of at least 4 or 5 bases that are identical to the target sequence, preferably continuous stretches of at least 6 or 7 bases that are identical to the target sequence, more preferably continuous stretches of at least 8 or 9 bases that are identical to the target sequence. The oligomer may have stretches of at least 10, 11, 12, 13, 14, 15, 16, 17, 18, 19, 20, 21, 22, 23, 24, 25 or 26 bases that are identical to the target sequence. The remaining stretches of oligomer sequence may be intermittently identical with the target sequence; for example, the remaining sequence may have an identical base, followed by a non-identical base, followed by an identical base. Alternatively (or as well) the oligomer sequence may have several stretches of identical sequence (for example 3, 4, 5 or 6 bases) interspersed with stretches of less than perfect homology. Such sequence mismatches will preferably have no or very little loss of splice switching activity.

The term “modulate” or “modulates” includes to “increase” or “decrease” one or more quantifiable parameters, optionally by a defined and/or statistically significant amount. The terms “increase” or “increasing,” “enhance” or “enhancing,” or “stimulate” or “stimulating” refer generally to the ability of one or AONs or compositions to produce or cause a greater physiological response (i.e., downstream effects) in a cell or a subject relative to the response caused by either no AON or a control compound. The terms “decreasing” or “decrease” refer generally to the ability of one or AONs or compositions to produce or cause a reduced physiological response (i.e., downstream effects) in a cell or a subject relative to the response caused by either no AON or a control compound.

Relevant physiological or cellular responses (in vivo or in vitro) will be apparent to persons skilled in the art, and may include increases in the exclusion of specific exons in a RAGE-coding pre-mRNA, decreases in the amount of RAGE-coding pre-mRNA or decreases in the expression of functional RAGE protein in a cell, tissue, or subject in need thereof. An “increased” or “enhanced” amount is typically a statistically significant amount, and may include an increase that is 1.1, 1.2, 2, 3, 4, 5, 6, 7, 8, 9, 10, 15, 20, 30, 40, 50 or more times (e.g., 500, 1000 times) (including all integers and decimal points in between and above 1, e.g., 1.5, 1.6, 1.7, 1.8) the amount produced by no AON (the absence of an agent) or a control compound. The term “reduce” or “inhibit” may relate generally to the ability of one or more AONs or compositions to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of a disease such as cancer, neurodegenerative diseases, lung disorders, and other inflammatory diseases. A “decrease” in a response may be statistically significant as compared to the response produced by no AON or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

The length of an AON may vary, as long as it is capable of binding selectively to the intended location within the pre-mRNA molecule. The length of such sequences can be determined in accordance with selection procedures described herein. Generally, the AON will be from about 10 nucleotides in length, up to about 50 nucleotides in length. It will be appreciated, however, that any length of nucleotides within this range may be used in the method. Preferably, the length of the AON is between 10 and 40, 10 and 35, 15 to 30 nucleotides in length or 20 to 30 nucleotides in length, most preferably about 25 to 30 nucleotides in length. For example, the oligomer may be 20, 21, 22, 23, 24, 25, 26, 27, 28, 29 or 30 nucleotides in length.

As used herein, an “AON” refers to a linear sequence of nucleotides, or nucleotide analogs, that allows the nucleobase to hybridize to a target sequence in an RNA by Watson-Crick base pairing, to form an oligonucleotide:RNA heteroduplex within the target sequence. The terms “AON”, “AON”, “oligomer” and “antisense compound” may be used interchangeably to refer to an oligonucleotide. The cyclic subunits may be based on ribose or another pentose sugar or, in certain embodiments, a morpholino group (see description of morpholino oligonucleotides below). Also contemplated are peptide nucleic acids (PNAs), locked nucleic acids (LNAs), and 2′-O-Methyl oligonucleotides, 2′-O-Methoxyethyl oligonucleotides, among other antisense agents known in the art.

Included are non-naturally-occurring AONs, or “oligonucleotide analogs”, including AONs or oligonucleotides having (i) a modified backbone structure, e.g., a backbone other than the standard phosphodiester linkage found in naturally-occurring oligo- and polynucleotides, and/or (ii) modified sugar moieties, e.g., morpholino moieties rather than ribose or deoxyribose moieties. Oligonucleotide analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). Preferred analogs are those having a substantially uncharged, phosphorus containing backbone.

One method for producing AONs is the methylation of the 2′ hydroxyribose position and the incorporation of a phosphorothioate backbone produces molecules that superficially resemble RNA but that are much more resistant to nuclease degradation, although persons skilled in the art of the methods of the invention will be aware of other forms of suitable backbones that may be useable in the objectives of the methods of the invention.

Increased splice-switching may also be achieved with alternative oligonucleotide chemistry. For example, the AON may be chosen from the list comprising: phosphoramidate or phosphorodiamidate morpholino oligomer (PMO); PMO-X; PPMO; peptide nucleic acid (PNA); a locked nucleic acid (LNA) and derivatives including alpha-L-LNA, 2′-amino LNA, 4′-methyl LNA and 4′-O-methyl LNA; ethylene bridged nucleic acids (ENA) and their derivatives; phosphorothioate oligomer; tricyclo-DNA oligomer (tcDNA); tricyclophosphorothioate oligomer; 2′O-Methyl-modified oligomer (2′-OMe); 2′-O-methoxy ethyl (2′-MOE); 2′-fluoro, 2′-fluroarabino (FANA); unlocked nucleic acid (UNA); thermostable twisted intercalating nucleic acid (TINA), hexitol nucleic acid (HNA); cyclohexenyl nucleic acid (CeNA); 2′-amino (2′-NH2); 2′-O-ethyleneamine or any combination of the foregoing as mixmers or as gapmers. To further improve the delivery efficacy, the above mentioned modified nucleotides are often conjugated with fatty acids/lipid/cholesterol/amino acids/carbohydrates/polysaccharides/nanoparticles etc. to the sugar or nucleobase moieties. These conjugated nucleotide derivatives can also be used to construct exon skipping AONs. Antisense oligonucleotide-induced splice modification of the human RAGE gene transcripts have generally used either oligoribonucleotides, PNAs, 2OMe or MOE modified bases on a phosphorothioate backbone. When alternative chemistries are used to generate the AONs for use in the methods of the invention, the uracil (U) of the sequences provided herein may be replaced by a thymine (T).

Included within the AONs for use in the methods of the present invention are non-naturally-occurring oligomers, or “oligonucleotide analogues,” including oligomers having (i) a modified backbone structure, e.g., a backbone other than the standard phosphodiester linkage found in naturally-occurring oligo- and polynucleotides, and/or (ii) modified sugar moieties, e.g., morpholino moieties rather than ribose or deoxyribose moieties. Oligomer analogues support bases capable of hydrogen bonding by Watson-Crick base pairing to standard polynucleotide bases, where the analogue backbone presents the bases in a manner to permit such hydrogen bonding in a sequence-specific fashion between the oligomer analogue molecule and bases in a standard polynucleotide (e.g., single-stranded RNA or single-stranded DNA). Preferred analogues are those having a substantially uncharged, phosphorus containing backbone.

Antisense oligonucleotides that do not activate RNase H can be made in accordance with known techniques (see, e.g., U.S. Pat. No. 5,149,797). Such AONs, which may be deoxyribonucleotide or ribonucleotide sequences, simply contain any structural modification which sterically hinders or prevents binding of RNase H to a duplex molecule containing the oligomer as one member thereof, which structural modification does not substantially hinder or disrupt duplex formation. Because the portions of the oligomer involved in duplex formation are substantially different from those portions involved in RNase H binding thereto, numerous AONs that do not activate RNase H are available. For example, such AONs may be oligomers wherein at least one, or all, of the inter-nucleotide bridging phosphate residues are modified phosphates, such as methyl phosphonates, methyl phosphorothioates, phosphoromorpholidates, phosphoropiperazidates boranophosphates, amide linkages and phosphoramidates. For example, every other one of the internucleotide bridging phosphate residues may be modified as described. In another non-limiting example, such AONs are molecules wherein at least one, or all, of the nucleotides contain a 2′ lower alkyl moiety (such as, for example, C₁-C₄, linear or branched, saturated or unsaturated alkyl, such as methyl, ethyl, ethenyl, propyl, 1-propenyl, 2-propenyl, and isopropyl). For example, every other one of the nucleotides may be modified as described.

Specific examples of preferred AONs for use in the methods of the present invention include oligomers containing modified backbones or non-natural inter-nucleoside linkages. As defined in this specification, oligomers having modified backbones include those that retain a phosphorus atom in the backbone and those that do not have a phosphorus atom in the backbone. For the purposes of this specification, and as sometimes referenced in the art, modified oligomers that do not have a phosphorus atom in their inter-nucleoside backbone can also be considered to be AONs.

In other preferred oligomer mimetics, both the sugar and the inter-nucleoside linkage, i.e., the backbone, of the nucleotide units are replaced with novel groups. The base units are maintained for hybridization with an appropriate nucleic acid target compound. One such oligomeric compound, an oligomer mimetic that has been shown to have excellent hybridization properties, is referred to as a peptide nucleic acid (PNA). In PNA compounds, the sugar-backbone of an oligomer is replaced with an amide containing backbone, in particular an aminoethylglycine backbone. The nucleo-bases are retained and are bound directly or indirectly to aza nitrogen atoms of the amide portion of the backbone.

Another preferred chemistry is the phosphorodiamidate morpholino oligomer (PMO) oligomeric compounds, which are not degraded by any known nuclease or protease. These compounds are uncharged, do not activate RNase H activity when bound to a RNA strand and have been shown to exert sustained splice modulation after in vivo administration (Summerton and Weller, Antisense Nucleic Acid Drug Development, 7, 187-197).

Modified oligomers may also contain one or more substituted sugar moieties. Oligomers may also include nucleobase (often referred to in the art simply as “base”) modifications or substitutions. Certain nucleobases are particularly useful for increasing the binding affinity of the oligomeric compounds as described herein. These include 5-substituted pyrimidines, 6-azapyrimidines, and N-2, N-6 and 0-6 substituted purines, including 2-aminopropyladenine, 5-propynyluracil, 5-propynylcytosine and 5-methylcytosine substitutions have been shown to increase nucleic acid duplex stability by 0.6-1.2° C., even more particularly when combined with 2′-O-methoxyethyl sugar modifications.

The activity of AONs and variants thereof can be assayed according to routine techniques in the art. For example, splice forms and expression levels of surveyed RNAs and proteins may be assessed by any of a wide variety of well-known methods for detecting splice forms and/or expression of a transcribed nucleic acid or protein. Non-limiting examples of such methods include RT-PCR of spliced forms of RNA followed by size separation of PCR products, nucleic acid hybridization methods e.g., Northern blots and/or use of nucleic acid arrays; nucleic acid amplification methods; immunological methods for detection of proteins; protein purification methods; and protein function or activity assays.

RNA expression levels can be assessed by preparing mRNA/cDNA (i.e., a transcribed polynucleotide) from a cell, tissue or organism, and by hybridizing the mRNA/cDNA with a reference polynucleotide, which is a complement of the assayed nucleic acid, or a fragment thereof. cDNA can, optionally, be amplified using any of a variety of polymerase chain reaction or in vitro transcription methods prior to hybridization with the complementary polynucleotide; preferably, it is not amplified. Expression of one or more transcripts can also be detected using quantitative PCR to assess the level of expression of the transcript(s).

The AONs for use in the methods of the present invention may provide induced splice-switching of the RAGE gene transcript, clinically relevant oligomer chemistries and delivery systems to direct RAGE splice manipulation to therapeutic levels, promotion of the non-signalling decoy-receptor RAGE mRNA splicoform (e.g. RAGE_v1, RAGE_v6, RAGE_v8, RAGE_v9 RAGE_v0, RAGE_v15, RAGE_v18, and RAGE_v19 mRNA) can be achieved, and therefore may be used to treat a RAGE-related respiratory (pulmonary) disease or disorder.

The AONs for use in the methods of the present invention may be conveniently made through the well-known technique of solid phase synthesis. Equipment for such synthesis is sold by several vendors including, for example, Applied Biosystems (Foster City, Calif.). One method for synthesising oligomers on a modified solid support is described in U.S. Pat. No. 4,458,066.

Any other means for such synthesis known in the art may additionally or alternatively be employed. It is well known to use similar techniques to prepare oligomers such as the phosphorothioates and alkylated derivatives. In one such automated embodiment, diethyl-phosphoramidites are used as starting materials and may be synthesized as described by Beaucage, et al., (1981) Tetrahedron Letters, 22:1859-1862.

The AONs for use in the methods of the present invention are synthesised in vitro and do not include antisense compositions of biological origin, or genetic vector constructs designed to direct the in vivo synthesis of AONs.

### Administration, Dosage and Formulation

In any aspect, the subject may have received, or may be receiving a treatment administered to their respiratory tract, wherein the treatment increases endogenous soluble RAGE. The treatment may be for a respiratory (pulmonary) disorder or disease associated with, or caused by, RAGE which is selected from the group: Acute upper respiratory infections, rhinitis, nasopharyngitis, sinusitis, laryngitis, influenza and pneumonia, acute bronchitis, acute bronchiolitis, asthma, chronic obstructive pulmonary disease (COPD), bronchiectasis, emphysema, chronic lung diseases due to external agents, Acute Respiratory Distress Syndrome (ARDS), pulmonary eosinophilia, and pleuritic, lung trauma and recovery from lung injury, trauma and surgery.

The term ‘respiratory’ refers to the process by which oxygen is taken into the body and carbon dioxide is discharged, through the bodily system including the nose, throat, larynx, trachea, bronchi and lungs.

As used herein, the upper respiratory tract may include the following regions: nose and nasal passages, paranasal sinuses, the pharynx, and the portion of the larynx above the vocal folds (cords). Typically, the lower respiratory tract includes any one of more of the following regions: portion of the larynx below the vocal folds, trachea, bronchi and bronchioles. The lungs can be included in the lower respiratory tract and include the respiratory bronchioles, alveolar ducts, alveolar sacs, and alveoli.

The term ‘respiratory disease’ or ‘respiratory condition’ refers to any one of several ailments that involve inflammation and affect a component of the respiratory system including the upper (including the nasal cavity, pharynx and larynx) and lower respiratory tract (including trachea, bronchi and lungs).

A symptom of respiratory disease may include cough, excess sputum production, a sense of breathlessness or chest tightness with audible wheeze. Exercise capacity may be quite limited. In asthma the FEV1.0 (forced expiratory volume in one second) as a percentage of that predicted nomographically based on weight, height and age, may be decreased as may the peak expiratory flow rate in a forced expiration. In COPD the FEV1.0 as a ratio of the FVC is typically reduced to less than 0.7. The impact of each of these conditions may also be measured by days of lost work/school, disturbed sleep, requirement for bronchodilator drugs, or requirement for glucocorticoids including oral glucocorticoids.

The terms “treatment” or “treating” of a subject includes the application or administration of an AON as described herein with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

The methods of the present invention may provide AONs as described herein, that are adapted to aid in the prophylactic or therapeutic treatment, prevention or amelioration of symptoms of a disease such as a RAGE expression related disease or pathology in a form suitable for delivery to a patient.

Although the methods of the present invention finds application in humans, the methods of the invention is also useful for therapeutic veterinary purposes. The invention is useful for domestic or farm animals such as cattle, sheep, horses and poultry; for companion animals such as cats and dogs; and for zoo animals.

In certain embodiments, the AONs as described herein can be delivered by pulmonary or nasal routes (e.g., via nebulised saline incorporating the AONs). The highest endogenous expression of RAGE mRNA in the healthy human tissues is found in the lung and is accessible via the airways. Inhaled oligonucleotides are an emerging therapeutic modality for respiratory diseases. The airways are uniquely lined with pulmonary surfactants, which are primarily composed of zwitterionic lipids. These surfactant lipids possess cationic properties at the pH of the respiratory tract. When anionic oligonucleotides are inhaled, they tend to be adsorbed by the surfactants, resulting in reformulated particles that have been hypothesised to be efficiently taken up by bronchial and alveolar epithelial cells into the pulmonary cells. Of note, AONs have been shown to be able to withstand the nebulization process.

The AONs as described herein may be in compositions formulated for administration to the lower respiratory tract only. Limitation to the lower respiratory tract may be achieved by an amount, particularly volume and composition of form ie. particle size, physical form whether dry powder or solution droplet, of composition that would otherwise be administered to the upper respiratory tract. Alternatively, the AONs as described herein may be administered via a device that ensures retention in the lower respiratory tract only.

The AONs as described herein may be formulated for intranasal administration, including dry powder, sprays, mists, or aerosols. This may be particularly preferred for treatment of a respiratory infection.

Suitable formulations, wherein the carrier is a liquid, for administration, as for example, a nasal spray or as nasal drops, include aqueous or oily solutions of the active ingredient. Alternatively, the AONs as described herein may be provided as a dry powder and administered to the upper respiratory tract only as defined herein.

The selection of appropriate carriers depends upon the particular type of administration that is contemplated. For administration via the upper respiratory tract, e.g., the nasal mucosal surfaces, the compound can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2 (Remington's, Id. at page 1445). Of course, the ordinary artisan can readily determine a suitable saline content and pH for an innocuous aqueous carrier for nasal and/or upper respiratory administration.

Other ingredients, such as art known preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odour for the formulation. For nasal administration of solutions or suspensions according to the methods of the invention, various devices are available in the art for the generation of drops, droplets and sprays. For example, an AON as described herein can be administered into the nasal passages by means of a simple dropper (or pipet) that includes a glass, plastic or metal dispensing tube from which the contents are expelled drop by drop by means of air pressure provided by a manually powered pump, e.g., a flexible rubber bulb, attached to one end.

The delivery of a therapeutically useful amount of AONs may be achieved by methods previously published. For example, intracellular delivery of the AON may be via a composition comprising an admixture of the AON and an effective amount of a block copolymer. An example of this method is described in US patent application US20040248833. Other methods of delivery of AONs to the nucleus are described in Mann C J et al. (2001) Proc, Natl. Acad. Science, 98(1) 42-47, and in Gebski et al. (2003) Human Molecular Genetics, 12(15): 1801-1811. A method for introducing a nucleic acid molecule into a cell by way of an expression vector either as naked DNA or complexed to lipid carriers, is described in U.S. Pat. No. 6,806,084.

The AONs for use in the methods of the present invention may be administered in an effective amount. The phrase ‘therapeutically effective amount’ or ‘effective amount’ generally refers to an amount of an AON, a pharmaceutically acceptable salt, polymorph or prodrug thereof as described herein that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. Undesirable effects, e.g. side effects, are sometimes manifested along with the desired therapeutic effect, hence, a practitioner balances the potential benefits against the potential risks in determining what is an appropriate “effective amount”.

The exact amount required will vary from subject to subject, depending on the species, age and general condition of the subject, mode of administration and the like. Thus, it may not be possible to specify an exact “effective amount”. However, an appropriate “effective amount” in any subject case may be determined by one of ordinary skill in the art using only routine experimentation. In one aspect, the dose administered to a subject is any dose that reduces viral load.

In any embodiment, an AON, as described herein, is administered in an amount and manner effective to result in a peak blood concentration of at least 200-400 nM AON. Typically, one or more doses of AON are administered, generally at regular intervals, for a period of about one to two weeks.

In any embodiment the AON is administered in an amount of equal to, or greater than, 0.024 mg/kg. In any embodiment the AON is administered in an amount of equal to, or greater than 0.24 mg/kg. In any embodiment the AON is administered in amount of equal to, or less than, 0.5 mg/kg.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of subject oligomers, and can generally be estimated based on EC50s found to be effective in in vitro and in vivo animal models.

The methods of the invention extends also to a combination of two or more AONs capable of binding to a selected target to induce exon exclusion in a RAGE gene transcript. The combination may be a cocktail of two or more AONs, a construct comprising two or more or two or more AONs joined together for use in an AON-based therapy. The combination of AONs is preferably a combination of SEQ ID NO: 11 and 10, or SEQ ID NO: 11 and 13.

Kits are also provided for use in the methods of the invention, in particular to measuring the level of endogenous soluble RAGE as described herein in a blood sample obtained from an subject, which kit comprises at least one probe to detect said endogenous soluble RAGE together with instructions for its use, wherein the instructions for use describe a method of the invention. The kit may further comprise a AON as described herein for use according to the methods of the invention.

The contents of the kit can be lyophilized and the kit can additionally contain a suitable solvent for reconstitution of the lyophilized components. Subject components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

As used herein the term “derived” and “derived from” shall be taken to indicate that a specific integer may be obtained from a particular source albeit not necessarily directly from that source.

As used herein, the singular forms “a,” “an” and “the” include plural references unless the context clearly dictates otherwise.

Other than in the operating example, or where otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the specification and claims are approximations that may vary depending upon the desired properties sought to be obtained by the methods of the present invention. Hence “about 80%” means “about 80%” and also “80%”. At the very least, each numerical parameter should be construed in light of the number of significant digits and ordinary rounding approaches.

Notwithstanding that the numerical ranges and parameters setting forth the broad scope of the methods of the present invention are approximations, the numerical values set forth in the specific examples are reported as precisely as possible. Any numerical value, however, inherently contains certain errors necessarily resulting from the standard deviation found in their respective testing measurements

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

It will be understood that the invention disclosed and defined in this specification extends to all alternative combinations of two or more of the subject features mentioned or evident from the text or drawings. All of these different combinations constitute various alternative aspects of the invention.

### EXAMPLES

### Example 1—Materials and Methods

### Animals

Studies undertaken to explore the duration of efficacy of antisense oligonucleotides targeting mouse RAGE pre-RNA were undertaken in healthy pathogen-free male C57Bl/6 mice (8-10 weeks old). The animals were housed in sterile passive micro-isolators at a constant 20° C. temperature on a 12-h day/night cycle and fed irradiated Barastoc mouse feed with irradiated tap water allowed ad libitum. Mouse weights were monitored throughout the experiment. Throughout the study animals were given access to mouse chow and water ad libitum. All experiments were approved by local animal ethics committee and conducted in accordance with the Guide for the Care and Use of Laboratory Animals published by the US National Institutes of Health (NIH Publication No. 85-23, revised 1996).

### Intratrachael Treatment

Mice were anaesthetised using a mixture of ketamine/xylazine (90 mg/10 mg/kg body weight via intraperitoneal injection) and intratracheal treatment undertaken when there was no reflex response (limb withdrawal to the hind paw). The tracheal opening was directly visualised using an otoscope/speculum device placed carefully into the mouth. Fifty microlitres of the allocated sterile solution was administered directly into the trachea through a blunted needle and high pressure syringe. Following the procedure, mice were returned to their home cage to recover on warmed blanket. Mice were monitored until full recovery was observed, after which they were provided fluids in the form of a moistened tissue and moistened food pellets, along with dry food pellets on the floor of the cage and returned to the housing room. Mice were then followed for 3, 7, 14, or 28 days at which time they were humanely euthanised using CO2 narcosis.

### Aerosolised Treatment

Mice were anaesthetised using a mixture of ketamine/xylazine (90 mg/10 mg/kg body weight via intraperitoneal injection) and intratracheal treatment undertaken when there was no reflex response (limb withdrawal to the hind paw). The tracheal opening was directly visualised using an otoscope/speculum device placed carefully into the mouth. Fifty microlitres of the allocated sterile solution was administered directly into the trachea through an aerosoliser micro-sprayer (Penn-Century IA-1C) connected to a high-pressure syringe (Penn-Century FMJ-250). Between each solution, the microsprayer and syringe was flushed three times with 250 μl of sterile ultrapure water and once with 250 μl sterile saline. Following the procedure, mice were returned to their home cage to recover on paper towel and provided warmth by placing half of the cage on a heat pad. Mice were monitored until full recovery was observed, after which they were provided fluids in the form of a moistened tissue and moistened food pellets, along with dry food pellets on the floor of the cage and returned to the housing room. Mice were then followed for 7 days at which time they were humanely euthanised.

### Sample Collection and Harvesting

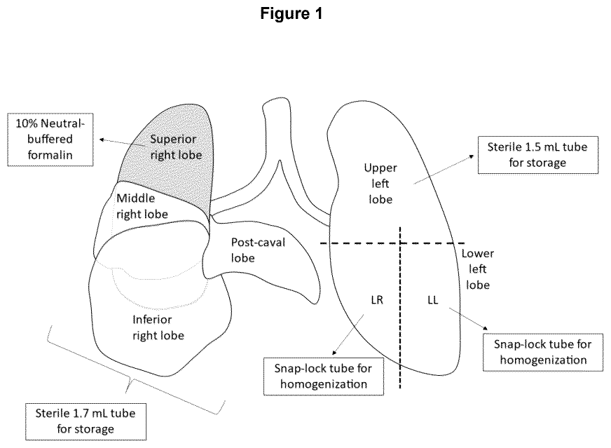

Immediately following humane euthanisation, all mice were exsanguinated by drawing blood from the inferior vena cava with a 19 G syringe lined with lithium heparin into a Microvette lithium/heparin tube (Sarstedt, Aust.). Blood was maintained at RT for a minimum of 15 minutes and centrifuged at 2000 g for 5 minutes at 20° C. Plasma was transferred into 1.5 mL snap-lock Eppendorf tubes, snap frozen in liquid nitrogen and stored at −80° C. Lungs were perfused to remove circulating blood. A 20 G needle was inserted into the right ventricle and 5-10 mL of chilled, sterile phosphate buffered saline was administered at a rate of 300 μl/second. The lungs were then excised as indicated in FIG. 1. The left lobe was divided into upper, lower left (LL) and lower right (LR) portions and snap frozen in liquid nitrogen.

Measuring Levels of esRAGE in Plasma and Lung Tissue

To measure the effect of interventions on the alternative splicing of RAGE pre-mRNA, the expression of RAGE mRNA splice variants was determined using real-time RT-qPCR, performed using the TaqMan system based on real-time detection of accumulated fluorescence (ABI Prism 7700, Perkin-Elmer Inc, PE Biosystems, Foster City, CA, USA) as previously utilized(43). For in vivo experiments, frozen lung tissue from the left lower lobe was used. RNA extraction cDNA synthesized was performed using the Trizol. Gene expression was estimated by RT-qPCR, performed using either the Taqman or SYBR Green (Sigma) system on the basis of real-time detection of accumulation of fluorescence (Applied Biosystems Q3 and Q5). Gene Expression was normalized to 18S mRNA and reported as fold change compared to the level of expression in control RNA treated cells, which were given an arbitrary value of 1. To define the expression of RAGE mRNA splice variants retaining exon 9b a probe was designed to span exon 9b. To define the expression of RAGE mRNA splice variants retaining exon 10, a PCR primer/probes spanning exon 8-10 were designed. To define the expression of all RAGE mRNA, PCR probe/primers spanning exon 8 and 11 were designed. A reduction in the signal of exon 8-10 of the cytosolic tail relative to exon 8-11 indicates that less full-length RAGE transcript (signalling capable) is produced by the cell.

Levels of murine extracellular soluble RAGE (esRAGE) in plasma and the lower right of the left lower lobe of the lung extracted in RIPA buffer were estimated using a commercial ELISA for murine esRAGE (ELISA Kit-MBS7606654-mybiosource). For lung tissue, results were standardised for protein content using the BCA assay (Pierce) (pg/60 ug protein). Plasma results were standardised to volume (pg/ml plasma).

### Statistical Analysis

Data shown are means±SEM. All datasets were determined to be normally distributed based on previous experiments and were also tested using the Kolmogorov-Smirnov test (α=0.05). Data were analysed using one-way ANOVA with Tukey's multiple comparison test using PRISM software (V6.0d, GraphPad, La Jolla, CA, USA), with P<0.05 considered statistically significant.

### Example 2—Plasma esRAGE Correlates with Lung Tissue esRAGE Following Intratracheal Treatment with ASO m79

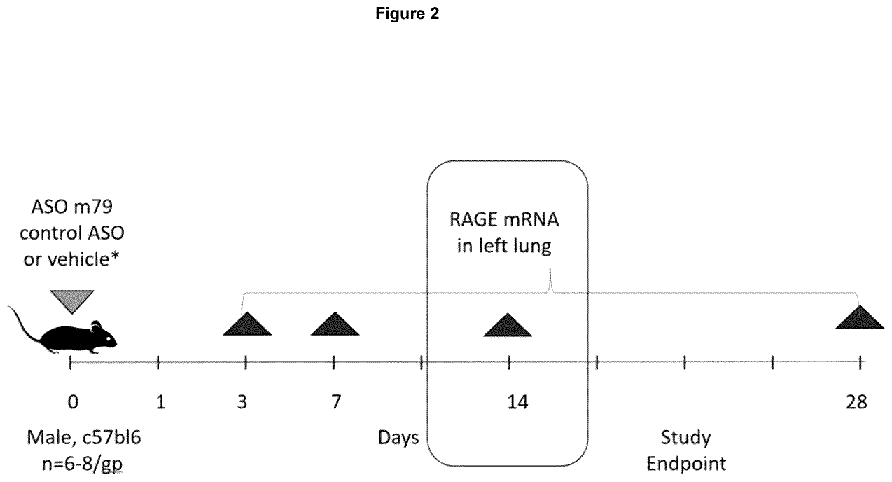

In this experiment, mice (6-8 per group) were treated with an intratracheal micro-spray of 50 ul of AON m79 (also referred to as ASO m79) (3 mg/kg; SEQ ID NO: 27) or a control oligonucleotide (GCAGUUGGCCCCUCCUC; SEQ ID NO: 33). ASO m79 is a 25-mer mouse antisense oligonucleotide that when administered to a cell promotes RAGE_v1 mRNA expression and therefore esRAGE production. AON m79 is an exemplary AON that is complementary to a pre-mRNA target region that has a 5′-most nucleotide at position 88 or 114 of exon 10 or that is between nucleotide positions 88 to 114 of exon 10. esRAGE levels were measured in the left lung tissue and plasma on day 14 (FIG. 2)

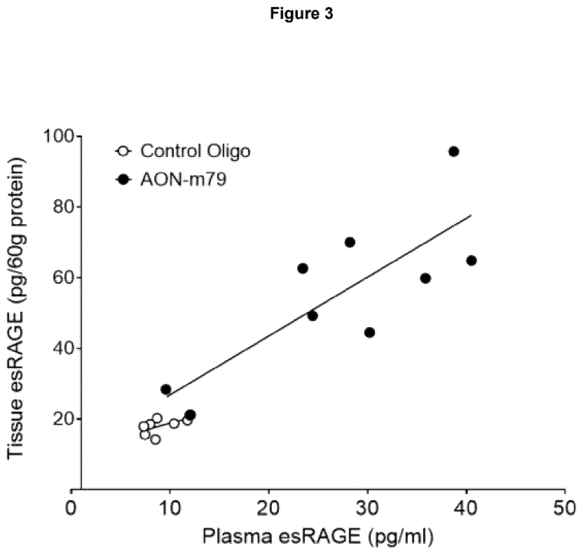

esRAGE in plasma at day 14 days correlated strongly with lung tissue esRAGE in the lung tissue. In contrast the control group did show a significant correlation between plasma and lung tissue esRAGE levels (FIG. 3).

This data suggest that esRAGE levels in the plasma can be used as a biomarker to determine the efficacy of ASO m79 treatment in the lung.

### Example 3—Plasma esRAGE Correlates with Lung Tissue esRAGE Following Intratracheal Treatment with ASO2

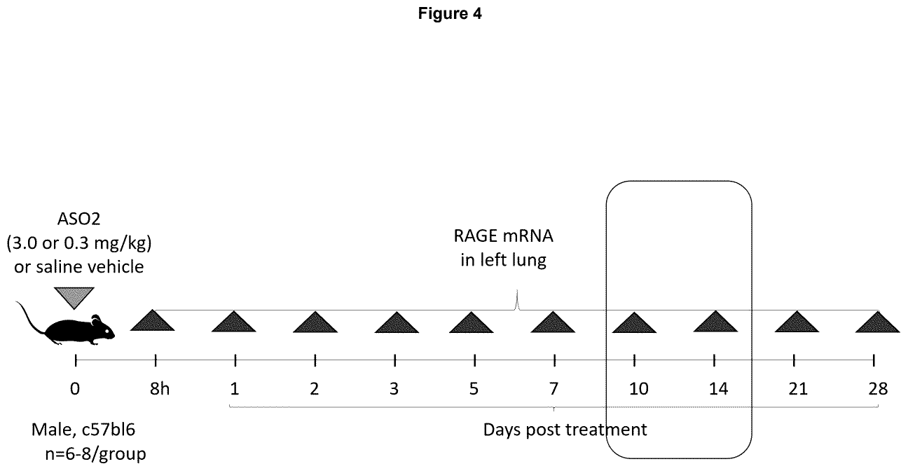

In this experiment, mice were treated with an intratracheal micro-spray of 50 ul of ASO2 (0.3 or 3.0; 8 per group) or saline (vehicle; 4/group) (FIG. 4). ASO2 is a human 18-mer antisense oligonucleotide with modified nucleotide bases that when administered to a cell promotes RAGE_v1 mRNA expression and therefore esRAGE production. esRAGE levels was measured in the lung tissue and plasma at day 10 and day 14 following treatment with ASO2.

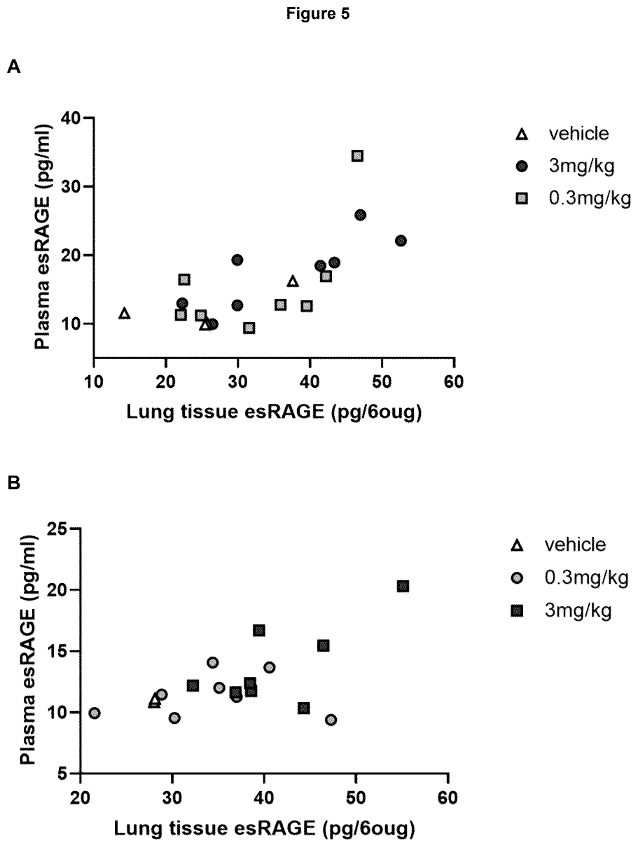

esRAGE in plasma correlated strongly with lung tissue esRAGE at day 10 and day 14 following single dose administration of with 3 mg/kg or 0.3 mg/kg of ASO2, p<0.01 (FIG. 5).

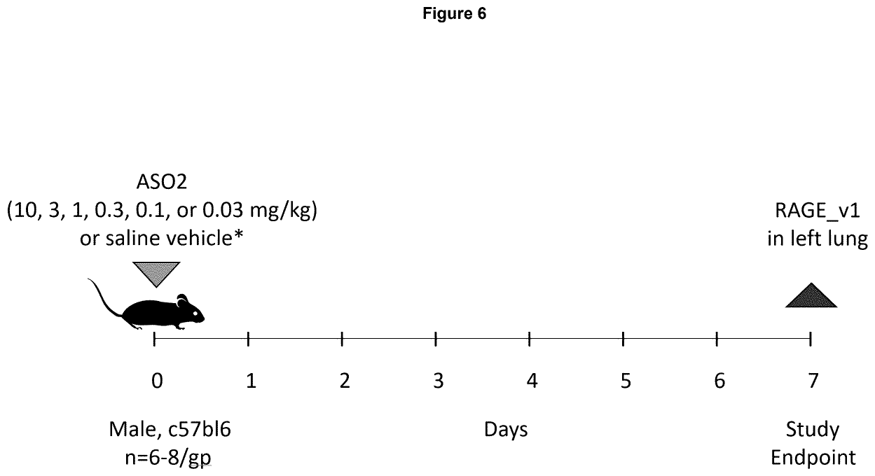

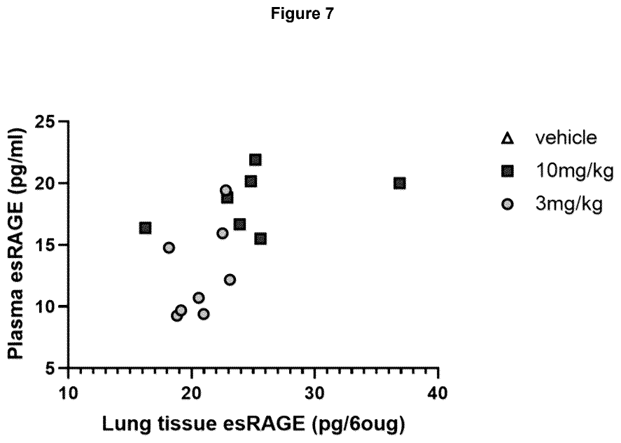

A similar statistically significant correlation was also observed when ASO2 was administered using an intratracheal micro-sprayer with doses of 10 mg/kg and 3 mg/kg (FIGS. 6 and 7).

Together these results suggest that plasma esRAGE can be used as a biomarker to determine the levels of esRAGE in the lung following treatment with an AON that modulates pre-mRNA RAGE splicing.

1. A method for determining endogenous soluble receptor for advanced glycation end products (RAGE) level in the lung of a subject, the method comprising:
determining the presence of endogenous soluble RAGE in a blood sample from a subject who has received or is receiving a treatment administered to their respiratory tract, wherein the treatment increases endogenous soluble RAGE,
wherein the level of endogenous soluble RAGE in the blood sample correlates with the endogenous soluble RAGE level in the lung of the subject.

2. A method for determining endogenous soluble receptor for advanced glycation end products (RAGE) level in a blood sample of a subject, the method comprising:
determining the presence of endogenous soluble RAGE in a blood sample from a subject who has received or is receiving a treatment administered to their respiratory tract, wherein the treatment increases endogenous soluble RAGE,
thereby determining endogenous soluble RAGE in the blood sample of the subject.

3. A method for determining the splicing of Receptor for Advanced Glycation End-products (RAGE) pre-mRNA resulting in the inclusion of exon 9b and/or exclusion of exon 10 in a subject, the method comprising:
determining the presence of endogenous soluble RAGE in a blood sample from a subject who has received or is receiving an antisense oligonucleotide (AON), that promotes splicing in the RAGE pre-mRNA resulting in the inclusion of exon 9b and/or exclusion of exon 10, to the respiratory tract,
wherein the presence of endogenous soluble RAGE in the blood sample indicates the splicing of Receptor for Advanced Glycation End-products (RAGE) pre-mRNA resulting in the inclusion of exon 9b and/or exclusion of exon 10 in the subject.

4. A method for determining the splicing of Receptor for Advanced Glycation End-products (RAGE) pre-mRNA resulting in the inclusion of exon 9b and/or exclusion of exon 10 in a subject, the method comprising:
providing a blood sample from a subject who has received or is receiving an antisense oligonucleotide (AON), that promotes splicing in the RAGE pre-mRNA resulting in the inclusion of exon 9b and/or exclusion of exon 10, to the respiratory tract,
measuring the level of endogenous soluble RAGE in the blood sample,
wherein the level of endogenous soluble RAGE in the blood sample indicates the splicing of RAGE pre-mRNA resulting in the inclusion of exon 9b and/or exclusion of exon 10 in a subject.

5. A method for determining the splicing of Receptor for Advanced Glycation End-products (RAGE) pre-mRNA resulting in the inclusion of exon 9b and/or exclusion of exon 10 in a subject, the method comprising:
providing a blood sample from a subject who has received or is receiving an antisense oligonucleotide (AON), that promotes splicing in the RAGE pre-mRNA resulting in the inclusion of exon 9b and/or exclusion of exon 10, to the respiratory tract,
measuring the level of endogenous soluble RAGE in the blood sample,
wherein the level of endogenous soluble RAGE in the blood sample above a threshold level indicates the splicing of RAGE pre-mRNA resulting in the inclusion of exon 9b and/or exclusion of exon 10 in a subject.

6. A method for determining the splicing of Receptor for Advanced Glycation End-products (RAGE) pre-mRNA resulting in the inclusion of exon 9b and/or exclusion of exon 10 in a subject, the method comprising:
providing a blood sample from a subject prior to receiving an antisense oligonucleotide (AON), that promotes splicing in the RAGE pre-mRNA resulting in the inclusion of exon 9b and/or exclusion of exon 10, to the respiratory tract,
providing a blood sample from the subject after receiving an AON, that promotes splicing in the RAGE pre-mRNA resulting in the inclusion of exon 9b and/or exclusion of exon 10, to the respiratory tract,
measuring the level of endogenous soluble RAGE in each of the blood samples,
wherein a higher level of endogenous soluble RAGE in the blood sample from the subject after receiving the AON to the respiratory tract compared to the level of endogenous soluble RAGE in the blood sample from the subject prior to receiving the AON to the respiratory tract indicates splicing of RAGE pre-mRNA resulting in the inclusion of exon 9b and/or exclusion of exon 10 in a subject.

7. A method for determining the splicing of Receptor for Advanced Glycation End-products (RAGE) pre-mRNA resulting in the inclusion of exon 9b and/or exclusion of exon 10 in a subject, the method comprising:
obtaining a blood sample from a subject prior to receiving, to the respiratory tract, an antisense oligonucleotide (AON) that promotes splicing in the RAGE pre-mRNA resulting in the inclusion of exon 9b and/or exclusion of exon 10,
obtaining a blood sample from the subject after receiving, to the respiratory tract, an AON that promotes splicing in the RAGE pre-mRNA resulting in the inclusion of exon 9b and/or exclusion of exon 10,
measuring the level of endogenous soluble RAGE in each of the blood samples,
wherein a higher level of endogenous soluble RAGE in the blood sample from the subject after receiving the AON to the respiratory tract compared to the level of endogenous soluble RAGE in the blood sample from the subject prior to receiving the AON indicates that splicing of RAGE pre-mRNA resulting in the inclusion of exon 9b and/or exclusion of exon 10 in the subject.

8. A method for determining whether a treatment/AON can increase endogenous soluble RAGE level in the respiratory tract, the method comprising:
administering a test treatment/AON to the respiratory tract of a subject,
obtaining a blood sample from the subject after the administration,
wherein the presence of, or an increase in the level of, endogenous soluble RAGE in the blood sample indicates the test treatment/AON increase endogenous soluble RAGE level in the respiratory tract.

9. The method of any one of claims 1 to 8, wherein the blood sample comprises or consists of whole blood, plasma or serum.

10. The method of any one of claims 2 to 9, wherein the blood sample was obtained at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, or at least about 28 days after the subject received antisense oligonucleotide (AON) that promotes splicing in the RAGE pre-mRNA resulting in the inclusion of exon 9b and/or exclusion of exon 10 to the respiratory tract.

11. The method of any one of claims 2 to 10, wherein the AON promotes the production of endogenous soluble RAGE by promoting the inclusion of exon 9b and/or the exclusion (e.g. skipping) of exon 10.

12. The method of any one of claims 2 to 11, wherein the AON results in an increase in the level of RAGE_v1, RAGE_v6, RAGE_v8, RAGE_v9 RAGE_v10, RAGE_v5, RAGE_v18, and RAGE_v19mRNA, preferably an increase in the level of RAGE_v1, RAGE_v6, RAGE_v8, RAGE_v9 RAGE_v10, RAGE_v15, RAGE_v18, and RAGE_v19 mRNA in one or more tissues of the respiratory tract.

13. The method of any one of claims 2 to 12, wherein the AON is administered to the total respiratory tract, the upper respiratory tract or the lower respiratory tract.

14. The method of any one of claim 13, wherein the AON is administered to the lower respiratory tract.

15. The method of claim 14, wherein the AON is administered to one or more of the following regions: the portion of the larynx below the vocal folds, trachea, bronchi and bronchioles.

16. The method of claim 14, wherein the AON is administered to the lungs.

17. The method of any one of claims 2 to 16, wherein the AON is administered as an aerosol.

18. The method of any one of claims 2 to 17, wherein the AON is 10 to 50 nucleotides comprising a targeting sequence complementary to a region near or within an intron of the RAGE pre-mRNA.

19. The method of any one of claims 2 to 17, wherein the AON is 10 to 50 nucleotides comprising a targeting sequence complementary or adjacent to a splice site of the RAGE pre-mRNA.

20. The method of any one of claims 2 to 17, wherein the AON is 10 to 50 nucleotides comprising a targeting sequence complementary or adjacent to cis-acting RNA elements in the pre-mRNA of RAGE that act as enhancers or silencers, that, when bound by an elements of the splicosome modulates the splicing of a nearby exon.

21. The method of any one of claims 2 to 17, wherein the AON is 10 to 50 nucleotides comprising a targeting sequence complementary to RAGE pre-mRNA which modulates secondary structure of said mRNA to influence splice site selection.

22. The method of any one of claims 2 to 21, wherein the AON is an isolated or purified AON for inducing exclusion of one or more exonic sequences in the RAGE gene transcript or part thereof.

23. The method of any one of claims 2 to 22, wherein the AON is an isolated or purified AON for inducing retention of intronic sequences in the RAGE gene transcript or part thereof.

24. The method of any one of claims 2 to 23, wherein the AON comprises at least one modified nucleotide.

25. The method of any one of claims 2 to 24, wherein the AON is chemically-modified to prevent degradation of the pre-mRNA-AON complex.

26. The method of any one of claims 2 to 25, wherein the AON comprises one or more nucleotide modifications selected from the group consisting of:
phosphorodiamidate morpholino oligomers (PMO), 2′ O-methyl phosphorothioate oligonucleotides (2OMe), and 2′-O-methoxyethyl phosphorothioate oligonucleotides (2MOE), locked nucleic acid (LNA) modified AONs, thermostable twisted intercalating nucleic acid (TINA) and peptide nucleic acids (PNAs).

27. The method of any one of claims 2 to 26, wherein the AON is conjugated to at least one moiety to increase their delivery.

28. The method of claim 27, wherein the moiety is one or more of: cell-penetrating peptides (CPPs), vivo-morpholinos (VMO) and peptide phosphorodiamidate morpholino oligomers (PPMO).

29. The method of any one of claims 2 to 28, wherein the AON comprises, consists essentially of or consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% complementary to a target region of exon 10 of RAGE pre-mRNA over the entirety of the antisense oligonucleotide.

30. The method of claim 29, wherein the 5′-most nucleotide of the target region is nucleotide position 88 or 114 of exon 10 or is between nucleotide positions 88 to 114 of exon 10.

31. The method of claim 30, wherein the nucleotide positions of exon 10 correlate to the nucleotide sequence of SEQ ID NO: 32.

32. The method of any one of claims 2 to 31, wherein the AON may be 8 to 40 nucleotides in length, 15 to 25 nucleotides in length or 18 nucleotides in length.

33. The method of any one of claims 2 to 32, wherein the AON is selected from the group comprising the sequences set forth in any of Tables 1a-1d.

34. The method of claim 33, wherein the AON is selected from the list comprising: SEQ ID NO: 1-31 or a nucleotide sequence at least 85%, 90% or 95% identical thereto.

35. The method of claim 34, wherein the AON is SEQ ID NO: 11, 18, 19, or 20 or a nucleotide sequence at least 85%, 90% or 95% identical thereto.
